(19) United States
(12) Patent Application Publication
(10) Pub. No.: US 2006/0019945 A1
(43) Pub. Date: Jan. 26, 2006

(54) Granzyme b inhibitors

(76) Inventors: Kevin T. Chapman, Scotch Plains, NJ (US); Christopher A. Willoughby, Newsbury Park, CA (US); Yuang Cheng, Newbury Park, CA (US)

Correspondence Address: MERCK AND CO., INC, P O BOX 2000, RAHWAY, NJ, 07065-0907, US

(21) Appl. No.: 10/503,155

(22) PCT Filed: Jan. 31, 2003
(86) PCT No.: PCT/US03/02941

§ 371 Date: Jul. 28, 2004

Related U.S. Application Data
(60) Provisional application No. 60/354,251, filed on Feb. 4, 2002.

(51) Int. Cl. A61K 31/55; C07D 487/04

(52) U.S. Cl. 514/212.05; 540/522

(57) ABSTRACT

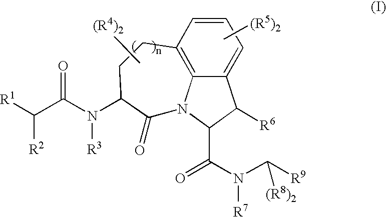

The present invention encompasses compounds of Formula (I) and pharmaceutically acceptable salts or hydrates thereof. The compounds are inhibitors of granzyme B and are useful for treating autoimmune and chronic inflammatory diseases. Pharmaceutical compositions and methods of use are also included.

### BACKGROUND OF THE INVENTION

Autoimmune diseases are diseases in which a specific immune response to self-molecules occurs, often leading to tissue and organ damage and dysfunction. The diseases can be organ-specific (e.g. Type I diabetes mellitus, thyroiditis, myasthenia gravis, primary biliary cirrhosis) or systemic in nature (e.g. systemic lupus erythematosus, rheumatoid arthritis, polymyositis, dermatomyositis, Sjogrenfs syndrome, scleroderma, and graft-vs.-host disease).

Apoptosis is a morphologically and biochemically distinct form of cell death that occurs in many different cell types during a wide range of physiologic and pathologic circumstances (reviewed in (Jacobson et al., 1997; Thompson, 1995; White, 1996)). Studies report that specific proteolysis catalyzed by a novel family of cysteine proteases is of critical importance in mediating apoptosis (Chinnaiyan and Dixit, 1996a; Martin and Green, 1995; Thornberry and Molineaux, 1995). These proteases (termed caspases), cleave downstream substrates after a consensus tetrapeptide sequence ending with aspartic acid. The caspases are synthesized as inactive precursors that require specific proteolytic cleavage after an aspartic acid residue for activation.

Granzyme B is a serine protease found in the cytoplasmic granules of cytotoxic T lymphocytes (CTL) and natural killer (NK) cells and has a similar requirement to caspases for aspartic acid in the substrate P₁ position (Odake et al., 1991; Poe et al., 1991). Studies have reported that granzyme B plays an important role in inducing apoptotic nuclear changes in target cells during granule exocytosis induced cytotoxicity (Darmon et al., 1996; Heusel et al., 1994; Sarin et al., 1997; Shresta et al., 1995; Talanian et al., 1997).

Granzyme B is described as catalyzing the cleavage and activation of several caspases (Chinnaiyan et al., 1996b; Darmon et al., 1995; Duan et al., 1996; Fernandes-Alnemri et al., 1996; Gu et al., 1996; Martin et al., 1996; Muzio et al., 1996; Quan et al., 1996; Sarin et al., 1997; Song et al., 1996a; Srinivasula et al., 1996; Talanian et al., 1997; Wang et al., 1996). Granzyme B also initiates caspase-independent pathways which contribute to target cell death. However, while several candidates for these additional pathways exist, they remain largely undefined (Sarin et al., 1997; Talanian et al., 1997).

One candidate pathway is the direct proteolysis of death substrates by granzyme B, although efficient non-caspase cellular substrates for this protease have not yet been identified. Initial studies have indicated that the cleavage of PARP, U1-70 kDa and lamin B observed during granzyme B-induced cell death is catalyzed by caspases, rather than directly by granzyme B (Darmon et al., 1995; Martinet al., 1996; Talanian et al., 1997), but the effects of granzyme B on other caspase substrates in vitro and during granule-induced cytotoxicity have not been extensively studied.

The present invention encompasses compounds that are inhibitors of granzyme B without inhibiting the caspases. The compounds are therefore useful for treating autoimmune and chronic inflammatory disease that may be specific to CTL-induced cytotoxicity.

### SUMMARY OF THE INVENTION

The present invention encompasses compounds of Formula I 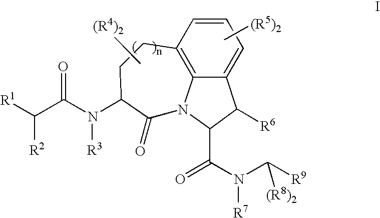
and pharmaceutically acceptable salts or hydrates thereof. The compounds are inhibitors of granzyme B and are useful for treating autoimmune and chronic inflammatory diseases. Pharmaceutical compositions and methods of use are also included.

### DETAILED DESCRIPTION OF THE INVENTION

The present invention encompasses compounds represented by Formula I: 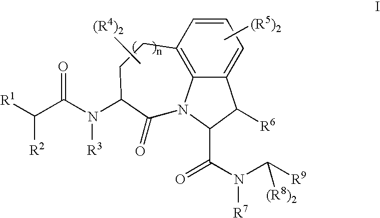
or a pharmaceutically acceptable salt or hydrate thereof, wherein:
n is 0, 1, or 2;
R¹ and R² are each independently selected from the group consisting of: hydrogen, C₁₋₆alkyl, C₁₋₆alkoxy, C₃₋₆cycloalkyl, aryl, HET and —N(R¹⁰)₂, wherein:
   (a) said C₁₋₆alkyl, C₁₋₆alkoxy and C₃₋₆cycloalkyl are optionally substituted with 1-3 substituents independently selected from the group consisting of halo and hydroxy; and
   (b) said aryl and BET are optionally substituted with 1-3 substituents independently selected from the group consisting of: halo, hydroxy and C₁₋₄alkyl, optionally substituted with 1-3 halo groups;
or R1 and R2 may be joined together with the carbon atom to which they are attached to form a five or six membered monocyclic ring, optionally containing 1-3 heteroatoms selected from the group consisting of: S, O and N(R¹⁰), wherein said ring is optionally substituted with 1-3 R¹⁰ groups,
   with the proviso that R¹ and R² are both not hydrogen;
each of R³ and R⁷ is independently selected from the group consisting of: hydrogen and C₁₋₄alkyl, optionally substituted with 1-3 halo groups;
each of R⁴, R⁵, R⁶ and R⁸ is independently selected from the group consisting of: hydrogen, halo, hydroxy and C₁₋₄alkyl, optionally substituted with 1-3 halo groups;
R⁹ is HET, optionally substituted with 1-3 substituents independently selected from the group consisting of: halo, hydroxy and C₁₋₄alkyl, optionally substituted with 1-3 halo groups;
R¹⁰ is selected from the group consisting of: hydrogen, C₁₋₄alkyl and —C(O)C₁₋₄alkyl, said —C(O)C₁₋₄alkyl optionally substituted with N(R¹¹)₂, HET and aryl, said aryl optionally substituted with 1-3 halo groups;
R¹¹ is selected from hydrogen and C₁₋₄alkyl, optionally substituted with 1-3 halo groups;
HET is a 5- to 10-membered aromatic, partially aromatic or non-aromatic mono- or bicyclic ring, containing 1-4 heteroatoms selected from O, S and N(R¹²), and optionally substituted with 1-2 oxo groups; and
R¹² is selected from the group consisting of: hydrogen and C₁₋₄alkyl, optionally substituted with 1-3 halo groups.

An embodiment of the invention encompasses the compound of Formula I wherein n is 0.

An embodiment of the invention encompasses the compound of Formula I wherein n is 1.

An embodiment of the invention encompasses the compound of Formula I wherein n is 2.

An embodiment of the invention encompasses the compound of Formula I wherein each of R³, R⁴, R⁵, R⁶, R⁷ and R⁸ is hydrogen.

An embodiment of the invention encompasses the compound of Formula I wherein BET is selected from the group consisting of: benzimidazolyl, benzofuranyl, benzopyrazolyl, benzotriazolyl, benzothiophenyl, benzoxazolyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, tetrazolyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, tetrahydrofuranyl, and tetrahydrothienyl, each optionally substituted with 1-2 substituents independently selected from the group consisting of: halo, oxo, hydroxy and C₁₋₄alkyl, optionally substituted with 1-3 halo groups.

An embodiment of the invention encompasses the compound of Formula I wherein R⁹ is selected from the group consisting of: pyridyl, pyrimidyl, pyrazinyl, pyridazinyl, imidazolyl, pyrazolyl, thiazolyl, oxazolyl, oxadiazolyl, isoxazolyl, 1,2,4-triazolyl, 1,2,3-triazolyl, and tetrazolyl, each optionally substituted with 1-3 substituents independently selected from the group consisting of: halo, hydroxy and C₁₋₄alkyl, optionally substituted with 1-3 halo groups.

An embodiment of the invention encompasses the compound of Formula I wherein R¹ and R² are each independently selected from the group consisting of: C₁₋₆alkyl, C₃₋₆cycloalkyl, phenyl, pyridyl, 2-oxopyrrolidine and —N(R¹⁰)₂, wherein:
(a) said C₁₋₆alkyl and C₃₋₆cycloalkyl optionally substituted with 1-3 groups independently selected from the group consisting of halo and hydroxy; and
(b) said phenyl, pyridyl and 2-oxopyrrolidine optionally substituted with 1-3 groups independently selected from the group consisting of: halo, hydroxy and C₁₋₄alkyl, optionally substituted with 1-3 halo groups; and
R¹⁰ is selected from the group consisting of: hydrogen, C₁₋₄alkyl, and —C(O)C₁₋₄alkyl, said —C(O)C₁₋₄alkyl optionally substituted with N(R¹¹)₂, pyrrolidine, piperidine, morpholine, benzothiophene and phenyl, said phenyl optionally substituted with 1-3 halo groups.

Within this embodiment of the invention is encompassed the compound of Formula I wherein n is 1. Also within this embodiment is encompassed the compound of Formula I wherein each of R³, R⁴, R⁵, R⁶, R⁷ and R⁸ is hydrogen. Also within this embodiment is encompassed the compound of Formula I wherein R⁹ is selected from the group consisting of: pyridyl, pyrimidyl, pyrazinyl, pyridazinyl, imidazolyl, pyrazolyl, thiazolyl, oxazolyl, oxadiazolyl, isoxazolyl, 1,2,4-triazolyl, 1,2,3-triazolyl, and tetrazolyl, each optionally substituted with 1-3 substituents independently selected from the group consisting of: halo, hydroxy and C₁₋₄alkyl, optionally substituted with 1-3 halo groups.

Another embodiment of the invention encompasses a compound of Formula II: 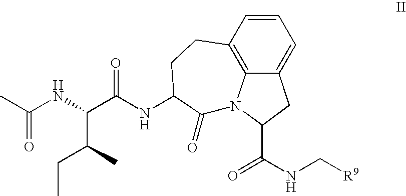
or a pharmaceutically acceptable salt or hydrate thereof, wherein:
R⁹ is selected from the group consisting of: benzimidazolyl, benzofuranyl, benzopyrazolyl, benzotriazolyl, benzothiophenyl, benzoxazolyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, tetrazolyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, tetrahydrofuranyl, and tetrahydrothienyl.

Within this embodiment is encompassed the compound of Formula I wherein R⁹ is selected from the group consisting of: pyridyl, pyrimidyl, pyrazinyl, pyridazinyl, imidazolyl, pyrazolyl, thiazolyl, oxazolyl, oxadiazolyl, isoxazolyl, 1,2,4-triazolyl, 1,2,3-triazolyl, and tetrazolyl.

Another embodiment of the invention encompasses a pharmaceutical composition comprising a compound of Formula I in combination with a pharmaceutically acceptable carrier.

Another embodiment of the invention encompasses a method of treating an immunoregulatory abnormality in a mammalian patient in need of such treatment comprising administering to said patient a compound in accordance with claim 1 in an amount that is effective for treating said immunoregulatory abnormality.

Another embodiment of the invention encompasses the above method wherein the immunoregulatory abnormality is an autoimmune or chronic inflammatory disease selected from the group consisting of: systemic lupus erythematosis, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmopathy, asthma, schleroderma and Sjogren's syndrome.

Another embodiment of the invention encompasses the above method wherein the immunoregulatory abnormality is bone marrow or organ transplant rejection or graft-versus-host disease.

Another embodiment of the invention encompasses the above method wherein the immunoregulatory abnormality is selected from the group consisting of: transplantation of organs or tissue, graft-versus-host diseases brought about by transplantation, autoimmune syndromes including rheumatoid arthritis, systemic lupus erythematosus, Hashimoto's thyroiditis, multiple sclerosis, myasthenia gravis, type I diabetes, uveitis, posterior uveitis, allergic encephalomyelitis, glomerulonephritis, post-infectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C₄ release, Behcet's disease, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, trauma, chronic bacterial infection, malignancy of lymphoid origin and acute and chronic lymphocytic leukemias and lymphomas.

Another embodiment of the invention encompasses the above method wherein the immunoregulatory abnormality is multiple sclerosis.

Another embodiment of the invention encompasses the above method wherein the immunoregulatory abnormality is rheumatoid arthritis.

Another embodiment of the invention encompasses the above method wherein the immunoregulatory abnormality is systemic lupus erythematosus.

Another embodiment of the invention encompasses the above method wherein the immunoregulatory abnormality is psoriasis.

Another embodiment of the invention encompasses the above method wherein the immunoregulatory abnormality is rejection of transplanted organ or tissue.

Another embodiment of the invention encompasses the above method wherein the immunoregulatory abnormality is inflammatory bowel disease.

Another embodiment of the invention encompasses the above method wherein the immunoregulatory abnormality is a malignancy of lymphoid origin.

Another embodiment of the invention encompasses the above method wherein the immunoregulatory abnormality is acute and chronic lymphocytic leukemias and lymphomas.

Another embodiment of the invention encompasses the above method wherein the immunoregulatory abnormality is selected from the group consisting of: schleroderma, autoimmune myositis, Sjogren's syndrome and type I diabetes.

Another embodiment of the invention encompasses a method of suppressing the immune system in a mammalian patient in need of immunosuppression comprising administering to said patient an immunosuppressing effective amount of a compound of Formula I.

Another embodiment of the invention encompasses a pharmaceutical composition comprising a compound which inhibits granzme B and does not substantially inhibit any caspase protease in combination with a pharmaceutically acceptable carrier.

Another embodiment of the invention encompasses a pharmaceutical composition comprising a compound which possesses a Ki of 500 nM or less for inhibiting granzyme B and possesses a Ki of 10,000 nM or more for inhibiting each of caspase-1 to caspase-13 in combination with a pharmaceutically acceptable carrier.

Another embodiment of the invention encompasses a method of treating an immunoregulatory abnormality in a mammalian patient in need of such treatment comprising administering to said patient a compound which inhibits granzme B and does not substantially inhibit any caspase protease in an amount that is effective for treating said immunoregulatory abnormality.

Another embodiment of the invention encompasses method of treating an immunoregulatory abnormality in a mammalian patient in need of such treatment comprising administering to said patient a compound which possesses a Ki of 500 nM or less for inhibiting granzyme B and possesses a Ki of 10,000 nM or more for inhibiting each of caspase-1 to caspase-13 in an amount that is effective for treating said immunoregulatory abnormality.

For purposes of this Specification, references to the activity of the compounds are as measured in the assays disclosed herein.

Exemplifying the invention are compounds of the following table:

TABLE 1

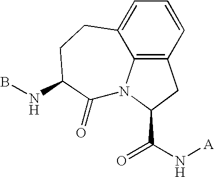

| EXAMPLE # | A | B |
|---|---|---|
| 1 | 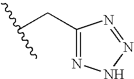 | 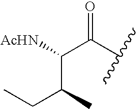 |
| 2 | 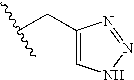 | 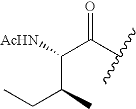 |
| 3 | 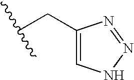 | 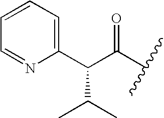 |
| 4 | 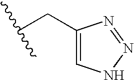 | 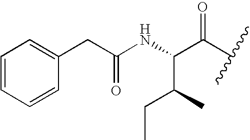 |
| 5 | 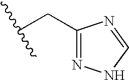 | 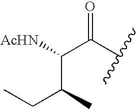 |
| 6 | 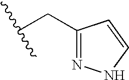 | 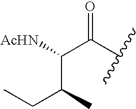 |
| 7 | 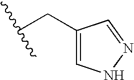 | 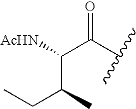 |
| 8 | 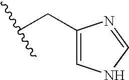 | 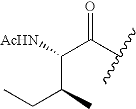 |
| 9 | 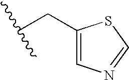 | 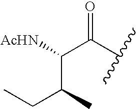 |
| 10 | 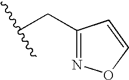 | 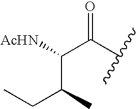 |
| 11 | 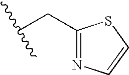 | 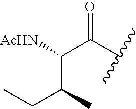 |
| 12 | 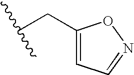 | 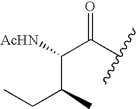 |
| 13 | 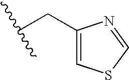 | 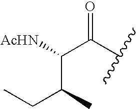 |
| 14 | 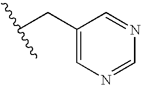 | 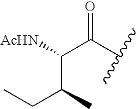 |
| 15 | 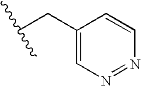 | 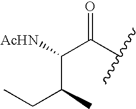 |
| 16 | 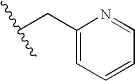 | 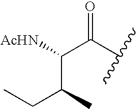 |
| 17 | 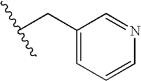 | 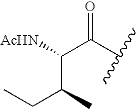 |
| 18 | 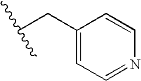 | 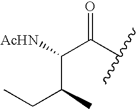 |
| 19 | 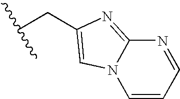 | 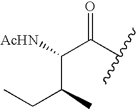 |
| 20 | 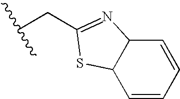 | 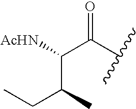 |
| 21 | 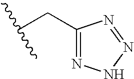 | 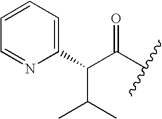 |
| 22 | 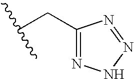 | 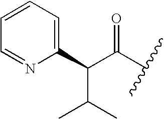 |
| 23 | 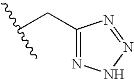 | 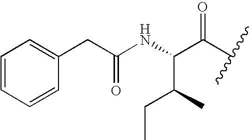 |
| 24 | 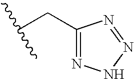 | 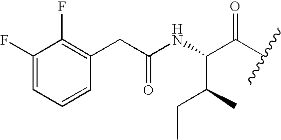 |
| 25 | 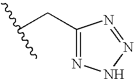 | 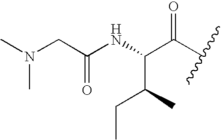 |
| 26 | 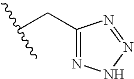 | 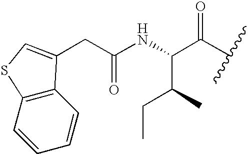 |
| 27 | 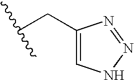 | 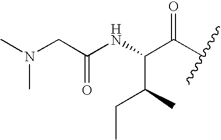 |
| 28 | 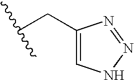 | 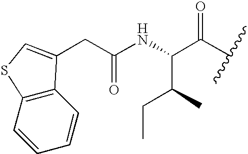 |
| 29 | 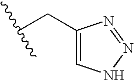 | 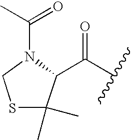 |
| 30 | 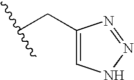 | 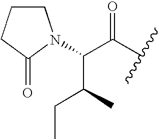 |
| 31 | 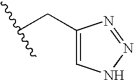 | 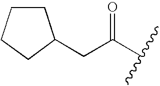 |
| 32 | 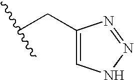 | 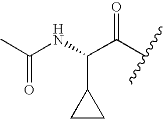 |
| 33 | 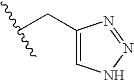 | 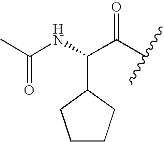 |

The invention is described using the following definitions unless otherwise indicated.

The term “halogen” or “halo” includes F, Cl, Br, and I.

The term “alkyl” means linear or branched structures and combinations thereof, having the indicated number of carbon atoms. Thus, for example, C₁₋₆alkyl includes methyl, ethyl, propyl, 2-propyl, s- and t-butyl, butyl, pentyl, hexyl, 1,1-dimethylethyl, cyclopropyl, cyclobutyl cyclopentyl and cyclohexyl.

The term “alkoxy” means alkoxy groups of a straight, branched or cyclic configuration having the indicated number of carbon atoms. C₁₋₆alkoxy, for example, includes methoxy, ethoxy, propoxy, isopropoxy, and the like.

The term “alkylthio” means alkylthio groups having the indicated number of carbon atoms of a straight, branched or cyclic configuration. C₁₋₆alkylthio, for example, includes methylthio, propylthio, isopropylthio, and the like.

The term “alkenyl” means linear or branched structures and combinations thereof, of the indicated number of carbon atoms, having at least one carbon-to-carbon double bond, wherein hydrogen may be replaced by an additional carbon-to-carbon double bond. C₂₋₆alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-carbon triple bond. C₃₋₆alkynyl, for example, includes, propenyl, 1-methylethenyl, butenyl and the like.

The term “cycloalkyl” means mono-, bi- or tri-cyclic structures, optionally combined with linear or branched structures, the indicated number of carbon atoms. Examples of cycloalkyl groups include cyclopropyl, cyclopentyl, cycloheptyl, adamantyl, cyclododecylmethyl, 2-ethyl-1-bicyclo[4.4.0]decyl, and the like.

The term “aryl” is defined as a mono- or bi-cyclic aromatic ring system and includes, for example, phenyl, naphthyl, and the like.

The term “aralkyl” means an alkyl group as defined above of 1 to 6 carbon atoms with an aryl group as defined above substituted for one of the alkyl hydrogen atoms, for example, benzyl and the like.

The term “aryloxy” means an aryl group as defined above attached to a molecule by an oxygen atom (aryl-O) and includes, for example, phenoxy, naphthoxy and the like.

The term “aralkoxy” means an aralkyl group as defined above attached to a molecule by an oxygen atom (aralkyl-O) and includes, for example, benzyloxy, and the like.

The term “arylthio” is defined as an aryl group as defined above attached to a molecule by an sulfur atom (aryl-S) and includes, for example, thiophenyoxy, thionaphthoxy and the like.

The term “aroyl” means an aryl group as defined above attached to a molecule by an carbonyl group (aryl-C(O)—) and includes, for example, benzoyl, naphthoyl and the like.

The term “aroyloxy” means an aroyl group as defined above attached to a molecule by an oxygen atom (aroyl-O) and includes, for example, benzoyloxy or benzoxy, naphthoyloxy and the like.

The term “HET” is defined as a 5- to 10-membered aromatic, partially aromatic or non-aromatic mono- or bicyclic ring, containing 1-4 heteroatoms selected from O, S and N, and optionally substituted with 1-2 oxo groups. Preferably, “HET” is a 5- or 6-membered aromatic or non-aromatic monocyclic ring containing 1-5 heteroatoms selected from O, S and N, for example, pyridine, pyrimidine, pyridazine, furan, thiophene, thiazole, oxazole, isooxazole and the like, or heterocycle is a 9- or 10-membered aromatic or partially aromatic bicyclic ring containing 1-5 heteroatoms selected from O, S, and N, for example, benzofuran, benzothiophene, indole, pyranopyrrole, benzopyran, quionoline, benzocyclohexyl, naphtyridine and the like. “HET” also includes the following: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, tetrazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl.

The term “treating” encompasses not only treating a patient to relieve the patient of the signs and symptoms of the disease or condition but also prophylactically treating an asymptomatic patient to prevent the onset or progression of the disease or condition. The term “amount effective for treating” is intended to mean that amount of a drug or pharmaceutical agent that will elicit the biological or medical response of a tissue, a system, animal or human that is being sought by a researcher, veterinarian, medical doctor or other clinician. The term also encompasses the amount of a pharmaceutical drug that will prevent or reduce the risk of occurrence of the biological or medical event that is sought to be prevented in a tissue, a system, animal or human by a researcher, veterinarian, medical doctor or other clinician.

For purposes of this specification, the following abbreviations have the indicated meanings:

| | |
|---|---|
| AcOH = | acetic acid |
| Alloc = | allyloxycarbonyl |
| APCI = | atmospheric pressure chemical ionization |
| BOC = | t-butyloxycarbonyl |
| CBZ = | carbobenzoxy |
| DCC = | 1,3-dicyclohexylcarbodiimide |
| DIBAL = | diisobutyl aluminum hydride |
| DIEA = | N,N-diisoproylethylamine |
| DMAP = | 4-(dimethylamino)pyridine |
| DMF = | dimethyl formamide |
| DTT = | dithiothreitol |
| EDCI = | 1-(3-dimethylaminopropyl)-3-ethylcarbodiimide |
| | hydrochloride |
| EDTA = | ethylenediaminetetraacetic acid, tetrasodium salt |
| | hydrate |
| ESI = | electrospray ionization |
| FAB = | fast atom bombardment |
| FMOC = | 9-fluorenylmethoxycarbonyl |
| HMPA = | hexamethylphosphoramide |
| HATU = | O-(7-Azabenzotriazol-1-yl)N,N,N′,N′- |
| | tetramethyluronium hexafluorophosphate |
| HOBT = | 1-hydroxybenzotriazole |
| HRMS = | high resolution mass spectrometry |
| ICl = | iodine monochloride |
| IBCF = | isobutyl chloroformate |
| KHMDS = | potassium hexamethyldisilazane |
| LDA = | lithium diisopropylamide |
| MCPBA = | metachloroperbenzoic acid |
| Ms = | methanesulfonyl = mesyl |
| MsO = | methanesulfonate = mesylate |
| NBS = | N-bromosuccinimide |
| NMM = | 4-methylmorpholine |
| PCC = | pyridinium chlorochromate |
| PDC = | pyridinium dichromate |
| Ph = | phenyl |
| PPTS = | pyridinium p-toluene sulfonate |
| pTSA = | p-toluene sulfonic acid |
| r.t. = | room temperature |
| rac. = | racemic |
| TFA = | trifluoroacetate |
| TfO = | trifluoromethanesulfonate = triflate |
| TLC = | thin layer chromatography |

Alkyl group abbreviations:

| | |
|---|---|
| Me = | methyl |
| Et = | ethyl |
| n-Pr = | normal propyl |
| i-Pr = | isopropyl |
| n-Bu = | normal butyl |
| i-Bu = | isobutyl |
| s-Bu = | secondary butyl |
| t-Bu = | tertiary butyl |

The compounds described herein are intended to include salts, enantiomers, esters and hydrates, in pure form and as a mixture thereof. Also, when a nitrogen atom appears, it is understood sufficient hydrogen atoms are present to satisfy the valency of the nitrogen atom.

While chiral structures are shown below, by substituting into the synthesis schemes an enantiomer other than the one shown, or by substituting into the schemes a mixture of enantiomers, a different isomer or a racemic mixture can be achieved. Thus, all such isomers and mixtures are included in the present invention.

The compounds described typically contain asymmetric centers and may thus give rise to diastereomers and optical isomers. The present invention is meant to comprehend such possible diastereomers as well as their racemic and resolved, enantiomerically pure forms and pharmaceutically acceptable salts thereof.

Some of the compounds described herein contain olefinic double bonds, and unless specified otherwise, are meant to include both E and Z geometric isomers.

The pharmaceutical compositions of the present invention comprise a compound of Formula I as an active ingredient or a pharmaceutically acceptable salt thereof in combination with a pharmaceutically acceptable carrier, and optionally other therapeutic ingredients. The term “pharmaceutically acceptable salts” refers to salts prepared from pharmaceutically acceptable bases including inorganic bases and organic bases. Representative salts derived from inorganic bases include aluminum, ammonium, calcium, copper, ferric, ferrous, lithium, magnesium, manganic salts, manganous, ammonium, potassium, sodium, zinc and the like. Particularly preferred are the calcium, magnesium, potassium, and sodium salts. Representative salts derived from pharmaceutically acceptable organic bases include salts of primary, secondary and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, salts may be prepared from pharmaceutically acceptable non-toxic acids, including inorganic and organic acids. Examples of such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric and tartaric acids.

In the discussion of methods of treatment that follows, reference to the compounds of Formula I are meant to also include the pharmaceutically acceptable salts.

By virtue of their Granzyme B inhibiting activity, the compounds of the present invention are useful for treating or preventing automimmune or chronic inflammatory diseases. The compounds of the present invention are useful to suppress the immune system in instances where immunosuppression is in order, such as in bone marrow, organ or transplant rejection, autoimmune and chronic inflammatory diseases, including systemic lupus erythematosis, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmopathy, asthma, schleroderma and Sjogren's syndrome.

More particularly, the compounds of the present invention are useful to treat or prevent a disease or disorder selected from the group consisting of: transplantation of organs or tissue, graft-versus-host diseases brought about by transplantation, autoimmune syndromes including rheumatoid arthritis, systemic lupus erythematosus, Hashimoto's thyroiditis, multiple sclerosis, myasthenia gravis, type I diabetes, uveitis, posterior uveitis, allergic encephalomyelitis, glomerulonephritis, post-infectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C₄ release, Behcet's disease, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, trauma, and chronic bacterial infection.

The magnitude of therapeutic dose of a compound of Formula I will, of course, vary with the nature of the severity of the condition to be treated and with the particular compound of Formula I and its route of administration and vary upon the clinician's judgement. It will also vary according to the age, weight and response of the individual patient. An effective dosage amount of the active component can thus be determined by the clinician after a consideration of all the criteria and using is best judgement on the patient's behalf. A representative dose will range from 0.001 mpk/d to about 100 mpk/d.

An ophthalmic preparations for ocular administration comprising 0.001-1% by weight solutions or suspensions of the compounds of Formula I in an acceptable ophthalmic formulation may be used.

Any suitable route of administration may be employed for providing an effective dosage of a compound of the present invention. For example, oral, parenteral and topical may be employed. Dosage forms include tablets, troches, dispersions, suspensions, solutions, capsules, creams, ointments, aerosols, and the like.

The compositions include compositions suitable for oral, parenteral and ocular (ophthalmic). They may be conveniently presented in unit dosage form and prepared by any of the methods well-known in the art of pharmacy.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration. In preparing the compositions for oral dosage form, any of the usual pharmaceutical media may be employed, such as, for example, water, alcohols, oils, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case or oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil emulsion. Such compositions may be prepared by any of the methods of pharmacy but all methods include the step of bringing into active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. For example, each dosage unit may contain from about 0.01 mg to about 1.0 g of the active ingredient.

Methods of Synthesis

The compounds of the present invention are prepared using the general procedures described below: 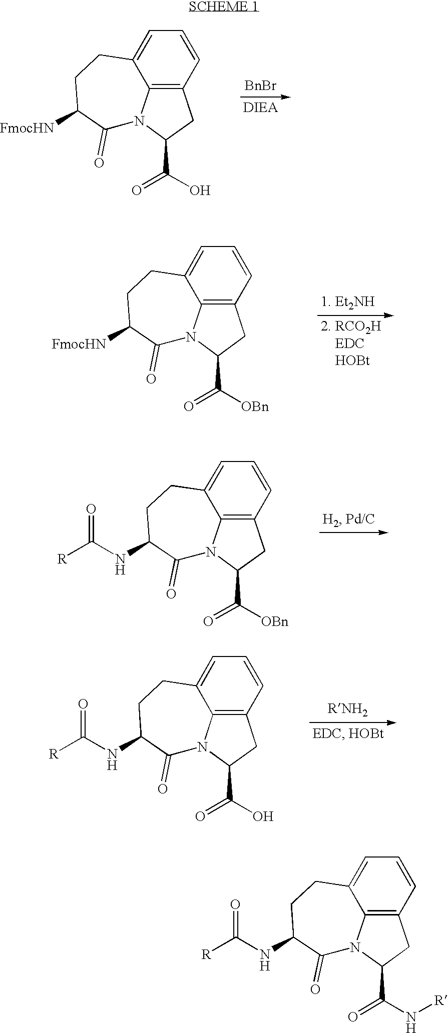
Commercially available (2S,5S)-5-{[(9H-fluoren-9-ylmethoxy)carbonyl]amino}-4-oxo-1,2,4,5,6,7-hexahydroazepino[3,2,1-hi]indole-2-carboxylic acid can be converted to the benzyl ester by reaction with benzyl bromide in the presence of a suitable base such as N,N-diisopropylethylamine or the like. Removal of the FMOC protecting group can be carried out with diethyl amine and the free amine can be subsequently coupled with a carboxylic acid using standard peptide coupling conditions. Removal of the benzyl protecting group can be conducted by catalytic hydrogenation with palladium or alternatively by hydrolysis with a suitable base such as lithium hydroxide. Coupling with the requisite amine can be accomplished with standard peptide coupling conditions such as with EDC/HOBt to afford the desired compounds.

Methods for preparing the compounds of this invention are further illustrated in the following examples. Alternative routes will be easily discernible to practitioners in the field.

### EXAMPLE 1

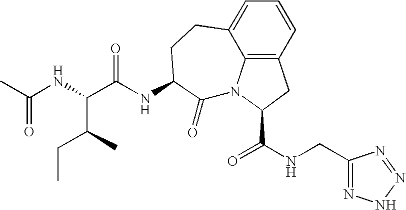

### (2S,5S)-5-[(N-acetyl-L-isoleucyl)amino]-4-oxo-N-(1H-tetraazol-5-ylmethyl)-1,2,4,5,6,7-hexahydroazepino[3,2,1-hi]indole-2-carboxamide

Step A: Benzyl (2S,5S)-5-{[(9H-fluoren-9-ylmethoxy)carbonyl]amino}-4-oxo-1,2,4,5,6,7-hexahydroazepino[3,2,1-hi]indole-2-carboxylate

A solution of (2S,5S)-5-{[(9H-fluoren-9-ylmethoxy)carbonyl]amino}-4-oxo-1,2,4,5,6,7-hexahydroazepino[3,2,1-hi]indole-2-carboxylic acid (1.5 grams, 3.2 mmol), N,N-diisopropylethyl amine (0.73 mL, 4.2 mmol) and benzyl bromide (0.46 mL, 3.8 mmol) were stirred in 4 mL DMF for 12 h. The mixture was diluted with EtOAc and washed with water (3×) and sat'd NaCl. The organic layer was dried over sodium sulfate and concentrated. Flash chromatography (3/1 hexanes/EtOAc) gave 1.6 grams (90%) product. ¹H NMR (500 MHz, CDCl₃) δ, 7.81 (d, 2H), 7.65 (d, 2H), 7.31-7.44 (6H), 7.12-7.21 (3H), 6.28 (d, 1H), 5.39 (d, 1H), 5.21 (dd, 2H), 4.42 (m, 2H), 4.37 (m, 1H), 4.28 (m, 1H, 3.53 (m, 2H), 3.38 (m, 1H), 3.17 (m, 2H), 2.41 (m, 1H), 2.18 (m, 1H).

Step B: Benzyl (2S,5S)-5-[(N-acetyl-L-isoleucyl)amino]-4-oxo-1,2,4,5,6,7-hexahydroazepino[3,2,1-hi]indole-2-carboxylate

A solution of benzyl (2S,5S)-5-{[(9H-fluoren-9-ylmethoxy)carbonyl]amino}-4-oxo-1,2,4,5,6,7-hexahydroazepino[3,2,1-hi]indole-2-carboxylate (1.6 grams, 2.8 mmol, from example 1 Step A) in 20 mL 1/1 diethylamine/dichloromethane was stirred for 2 h. The mixture was evaporated to dryness under vacuum then redissolved in 15 mL dichloromethane. Acetyl isoleucine (744 mg, 4.3 mmol), EDC (660 mg, 3.4 mmol) and HOBt (775 mg, 5.7 mmol) were added and the mixture was stirred for 10 h. The mixture was diluted with EtOAc and washed with 1 M HCl, sat'd sodium bicarbonate (2×) and sat'd NaCl. The organic layer was dried and concentrated. Flash chromatography (1/1 dichloromethane/ether) gave 1.2 grams (85%) of product. LC-MS (ESI) calc (M+H) 492.2, found. 492.3.

Step C: (2S,5S)-5-[(N-acetyl-L-isoleucyl)amino]-4-oxo-1,2,4,5,6,7-hexahydroazepino [3,2,1-hi]indole-2-carboxylic acid

A solution of benzyl (2S,5S)-5-[(N-acetyl-L-isoleucyl)amino]-4-oxo-1,2,4,5,6,7-hexahydroazepino[3,2,1-hi]indole-2-carboxylate (1.2 grams, 2.4 mmol, from example 1 Step B) in 40 mL methanol was stirred over palladium on carbon (260 mg, 10% wt., 0.24 mmol) under an atmosphere of hydrogen for 2 h. The mixture was filtered and concentrated to afford 0.93 grams (95%) of product. ¹ H NMR (500 MHz, DMSO) [symbol] , 8.21 (d, 1H), 7.93 (d, 1H), 7.15 (d, 1H), 7.08 (d, 1H), 6.95 (dd, 1H), 5.05 (d, 1H), 4.35 (m, 1H), 4.25 (t, 1H), 3.5 (dd, 1H), 3.2-3.4 (bs, 1H), 32.97-3.2 (m, 3H), 1.97-2.17 (m, 2H), 1.88 (s, 3H), 1.7-1.8 (m, 1H), 1.4-1.48 m, 1H), 1.08-1.17 (m, 1H), 0.9 (d, 3H), 0.82 (t, 3H).

Step D: (2S,5S)-5-[(N-acetyl-L-isoleucyl)amino]-N-(cyanomethyl)-4-oxo-1,2,4,5,6,7-hexahydroazepino[3,2,1-hi]indole-2-carboxamide

A mixture of (2S,5S)-5-[(N-acetyl-L-isoleucyl)amino]-4-oxo-1,2,4,5,6,7-hexahydroazepino [3,2,1-hi]indole-2-carboxylic acid (100 mg, 0.25 mmol, from example 1 Step C), aminoacetonitrile (17 mg, 0.3 mmol), EDC (72 mg, 0.357 mmol) and HOBt (68 mg, 0.5 mmol) were stirred together in 1 mL DMSO. After 1 h the mixture was purified by semi-prep HPLC (Column: YMC Pro-pack C18 5μ, 20×100 mm, gradient: 20%→80% acetonitrile/water with 0.1% TFA, 20 mL/min) to afford 86 mg (78%) of product. ¹H NMR (500 MHz, DMSO) [symbol] , 8.68 (d, 1H), 8.25 (d, 1H), 7.93 (d, 2H), 7.11 (d, 1H), 7.05 (d, 1H), 6.98 (dd, 1H), 5.02 (d, 1H), 4.37 (m, 1H), 4.23 (t, 1H), 4.12 (m, 2H), 3.44 (dd, 1H), 3.1 (m, 2H), 2.89 (d, 1H), 2.02-2.14 (m, 2H), 1.84 (s, 3H), 1.73 (m, 1H), 1.43 (m, 1H), 1.13 (m, 1H), 0.9 (d, 3H), 0.83 (t, 3H).

Step E: (2S,5S)-5-[(N-acetyl-L-isoleucyl)amino]-4-oxo-N-(1H-tetraazol-5-ylmethyl)-1,2,4,5,6,7-hexahydroazepino[3,2,1-hi]indole-2-carboxamide

A mixture of (2S,5S)-5-[(N-acetyl-L-isoleucyl)amino]-N-(cyanomethyl)-4-oxo-1,2,4,5,6,7-hexahydroazepino[3,2,1-hi]indole-2-carboxamide (27 mg, 0.06 mmol, from example 1 Step D), sodium azide (20 mg, 0.31 mmol) and triethylamine hydrochloride (51 mg, 0.37 ml) in 1 mL DMF was heated to 115° C. for 6 h. The product was isolated by semi-prep HPLC (Column: YMC Pro-pack C18 5 μ, 20×100 mm, gradient: 10%→70% acetonitrile/water with 0.1% TFA, 20 mL/min). ¹H NMR (500 MHz, DMSO) [symbol] , 8.74 (m, 1H), 8.21 (d, 1H), 7.92 (d, 1H), 7.08 (d, 1H), 7.04 (d, 1H), 6.95 (dd, 1H), 5.04 (d, 1H), 4.52 (ABX, 2H), 4.32 (m, 1H), 4.20 (t, 1H), 3.42 (dd, 1H), 3.05 (m, 2H), 2.98 (d, 1H), 2.03 (m, 2H), 1.84 (s, 3H), 1.73 (m, 1H), 1.42 (m, 1H), 1.11 (m, 1H), 0.86 (d, 3H), 0.81 (t, 3H). LC-MS (ESI) calc (M+H) 483.2, found. 483.3.

### EXAMPLE 2

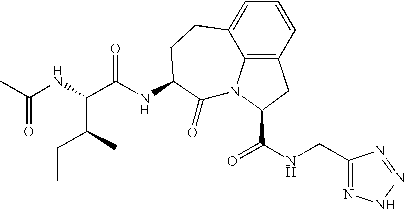

### (2S,5S)-5-[(N-acetyl-L-isoleucyl)amino]-4-oxo-N-(1H-1,2,3-triazol-4-ylmethyl)-1,2,4,5,6,7-hexahydroazepino[3,2,1-hi]indole-2-carboxamide

Step A: 1-(1H-1,2,3-triazol-4-yl)methylamine

Sodium azide (11.5 grams, 176 mmol), and propargyl bromide (27.7 grams, 80% wt solution in toluene, 186 mmol) were stirred in a mixture of 200 mL dioxane and 150 mL water for 10 h. The aqueous layer was separated and the organic layer was transferred to a pressure reactor. Concentrated ammonium hydroxide (200 mL) was added and the mixture was heated to 65° C. After 15 h the mixture was cooled and concentrated. Crystallization from dioxane/water afforded 5.2 grams (30%) of pure product. ¹H NMR (500 MHz, DMSO) [symbol] , 7.62 (s, 1H), 5.1-5.5 (bs, 3H), 3.79 (s, 2H).

Step B: (2S,5S)-5-[(N-acetyl-L-isoleucyl)amino]-4-oxo-N-(1H-1,2,3-triazol-4-ylmethyl)-1,2,4,5,6,7-hexahydroazepino[3,2,1-hi]indole-2-carboxamide

The compound was prepared according to the procedure in example 1 step D from (2S,5S)-5-[(N-acetyl-L-isoleucyl)amino]-4-oxo-1,2,4,5,6,7-hexahydroazepino[3,2,1-hi]indole-2-carboxylic acid (100 mg, 0.25 mmol, from example 1 Step C) and 1-(1H-1,2,3-triazol-4-yl)methylamine (50 mg, 0.5 mmol, from example 2 Step A). The product was purified by semi-prep HPLC (Column: YMC Pro-pack C18 5μ, 20×100 mm, gradient: 20%→40% acetonitrile/water with 0.1% TFA, 20 mL/min) to give 63 mg (52%). ¹H NMR (500 MHz, DMSO) [symbol] , 8.47 (m, 1H), 8.22 (d, 1H), 7.91 (d, 1H), 7.6 (bs, 1H), 7.08 (d, 1H), 7.04 (d, 1H), 6.95 (dd, 1H), 5.01 (d, 1H), 4.32 (m, 3H), 4.21 (t, 1H), 3.42 (dd, 1H), 3.07 (m, 2H), 2.95 (d, 1H), 2.03 (m, 2H), 1.84 (s, 3H), 1.72 (m, 1H), 1.42 (m, 1H), 1.11 (m, 1H), 0.87 (d, 3H), 0.81 (t, 3H). LC-MS (ESI) calc (M+H) 482.2, found. 482.3

### EXAMPLE 3

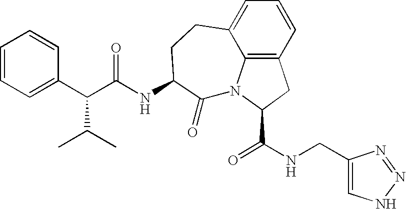

### (2S,5S)-5-{[(2R)-3-methyl-2-pyridin-2-ylbutanoyl]amino }-4-oxo-N-(1H-1,2,3-triazol-4-ylmethyl)-1,2,4,5,6,7-hexahydroazepino[3,2,1-hi]indole-2-carboxamide

Step A: Benzyl pyridin-2-ylacetate

2-pyridyl acetic acid hydrochloride (8 grams, 46 mmol), benzyl alcohol (19 mL, 184 mmol), EDC (13 grams, 69 mmol), N,N-diisopropylethyl amine (8 mL, 46 mmol) and DMAP (560 mg, 4.6 mmol) were combined in 150 mL dichloromethane and the mixture was stirred overnight. The mixture was diluted with EtOAc and extracted with 2M HCl (2×). The combined aqueous layers were neutralizes with solid sodium bicarbonate and extracted with EtOAc. The organic portion was dried with sodium sulfate and concentrated. Flash chromatography (2/1 hexanes/EtOAc) gave 8.6 grams (86%) of product. ¹H NMR (500 MHz, CDCl₃) [symbol] , 8.59 (d, 1H), 7.66 (dd, 1H), 7.3-7.4 (m, 6H), 7.2 (m, 1H), 5.19 (s, 2H), 3.96 (s, 2H).

Step B: Benzyl 3-methyl-2-pyridin-2-ylbutanoate

A solution of lithium hexamethyl disilazide (5.5 mL, 1.0 M in THF, 5.5 mmol) in and additional 15 mL THF was cooled to −78° C. under a nitrogen atmosphere. Benzyl pyridin-2-ylacetate (1.1 g, 5 mmol, from example 3 Step A) was added and the resulting mixture was stirred for 30 min. 2-Iodo propane (0.55 mL, 5.5 mmol) was added and the mixture was warmed to room temperature then stirred for 12 h. The reaction mixture was poured into sat'd ammonium chloride and extracted with EtOAc. The organic portion was dried over sodium sulfate and concentrated. Flash chromatography (5/1 hexane/EtOAc) gave 0.9 grams (67%) of product.

Step C: 3-Methyl-2-pyridin-2-ylbutanoic acid

A solution of benzyl 3-methyl-2-pyridin-2-ylbutanoate (0.9 grams, 3.3 mmol, from example 3 Step B) in 15 mL ethanol was stirred with palladium on carbon (350 mg, 0.33 mmol, 10% wt.) under 1 atmosphere of hydrogen. After 1.5 h the mixture was filtered through celite and concentrated to give 550 mg (93%) product. The product must be used immediately as it spontaneously looses CO₂. ¹H NMR (500 MHz, CD₃OD) [symbol] , 848 (d, 1H), 7.82 (dd, 1H), 7.55 (d, 1H), 7.34 (m, 1H), 3.42 (d, 1H), 2.42 (m, 1H), 1.1 (d, 3H), 0.73 (d, 3H).

Step D: Benzyl (2S,5S)-5-{[(2R)-3-methyl-2-pyridin-2-ylbutanoyl]amino}-4-oxo-1,2,4,5,6,7-hexahydroazepino[3,2,1-hi]indole-2-carboxylate

The title compound was prepared from 3-Methyl-2-pyridin-2-ylbutanoic acid (1.5 grams, 8.5 mmol, from example 3 Step C) and benzyl (2S,5S)-5-{[(9H-fluoren-9-ylmethoxy)carbonyl]amino}-4-oxo-1,2,4,5,6,7-hexahydroazepino[3,2,1-hi]indole-2-carboxylate (3.05 grams, 5.5 mmol, from example 1 Step A). Flash chromatography (5/1 hexanes/EtOAc) gave 1.7 grams (67%) of product. The desired diastereomer (faster eluting isomer) was isolated by chiral semi-prep HPLC (350 grams chiral-pak AD stationary phase eluting with isopropanol).

Step E: (2S,5S)-5-{[(2R)-3-methyl-2-pyridin-2-ylbutanoyl]amino}-4-oxo-1,2,4,5,6,7-hexahydroazepino[3,2,1-hi]indole-2-carboxylic acid

Benzyl (2S,5S)-5-{[(2R)-3-methyl-2-pyridin-2-ylbutanoyl]amino}-4-oxo-1,2,4,5,6,7-hexahydroazepino[3,2,1-hi]indole-2-carboxylate (550 mg, 1.1 mmol from example 3 Step D) was hydrogenated using the procedure described in example 1 Step C to afford 410 mg (92%) product. ¹H NMR (500 MHz, DMSO) [symbol] , 8.67 (d, 1H), 8.54 (d, 1H), 7.72 (dd, 1H), 7.43 (d, 1H), 7.25 (m, 1H), 7.1 (d, 1H), 7.03 (d, 1H), 6.96 (m, 1H), 5.03 (d, 1H), 4.37 (t, 1H), 3.48 (m, 1H), 2.4 (d, 1H), 2.98-3.15 (m, 3H), 2.38 (m, 1H), 1.95 (m, 2H), 1.05 (d, 3H), 0.65 (d, 3H).

Step F: (2S,5S)-5-{[(2R)-3-methyl-2-pyridin-2-ylbutanoyl]amino}-4-oxo-N-(1H-1,2,3-triazol-4-ylmethyl)-1,2,4,5,6,7-hexahydroazepino[3,2,1-hi]indole-2-carboxamide

The title compound was prepared from (2S,5S)-5-{[(2R)-3-methyl-2-pyridin-2-ylbutanoyl]amino}-4-oxo-1,2,4,5,6,7-hexahydroazepino[3,2,1-hi]indole-2-carboxylic acid (31 mg, 0.075 mmol, from example 3 Step E) and 1-(1H-1,2,3-triazol-4-yl)methylamine (15 mg, 0.15 mmol, from example 2 Step A) using the procedure described in example 2 Step B). ¹H NMR (500 MHz, DMSO) [symbol] , 8.64 (d, 1H), 8.47-8.51 (m, 2H), 7.70 (dd, 1H), 7.41 (d, 1H), 7.23 (m, 1H), 7.06 (d, 1H), 7.00 (d, 1H),6.93 (dd, 1H), 5.0 (d, 1H), 4.23-4.35 (h, 3H), 3.35-3.55 (m, 2H), 3.29 (m, 4H), 2.98-3.05 (m, 2H), 2.89 (d, H), 2.34 (m, 1H), 1.94-2.04 (m, 2H), 1.02 (d, 3H), 0.62 (d, 2H). LC-MS (ESI) calc (M+H) 487.2, found. 487.3

### EXAMPLE 4

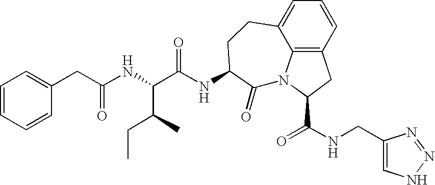

### (2S,5S)-4-oxo-5-{[N-(phenylacetyl)-L-isoleucyl]amino}-N-(1H-1,2,3-triazol-4-ylmethyl)-1,2,4,5,6,7-hexahydroazepino[3,2,1-hi]indole-2-carboxamide

Step A: N-(phenylacetyl)-L-isoleucine

A solution of isoleucine benzyl ester p-toluenesulfonate (3.9 grams, 10 mmol), phenyl acetic acid (1.4 grams, 10 mmol), EDC (2.3 grams, 12 mmol), HOBt (2.0 grams, 15 mmol) and N,N-diisopropyl ethylamine (1.9 mL, 11 mmol) were combined in 50 mL dichloromethane and the mixture was stirred for 3 h. The reaction mixture was diluted with EtOAc and washed with sat'd sodium bicarbonate (3×), water and sat'd NaCl. The organic layer was dried over sodium sulfate and concentrated. Flash chromatography (3/1 hexanes/EtOAc) gave 3.4 grams of product. The ester was dissolved in 50 mL methanol and stirred over palladium on carbon (1.0 grams, 1 mmol, 10% wt.) under a hydrogen atmosphere. After 2 h the mixture was filtered through celite and concentrated to afford 1.9 grams (75%) of the desired acid.

Step B: (2S,5S)-4-oxo-5-{[N-(phenylacetyl)-L-isoleucyl]amino}-1,2,4,5,6,7-hexahydroazepino[3,2,1-hi]indole-2-carboxylic acid

The title compound was prepared from N-(phenylacetyl)-L-isoleucine (100 mg, 0.40 mmol, from example 4 Step A) and benzyl (2S,5S)-5-{[(9H-fluoren-9-ylmethoxy)carbonyl]amino}-4-oxo-1,2,4,5,6,7-hexahydroazepino[3,2,1-hi]indole-2-carboxylate (150 mg, 0.27 mmol, from example 1 Step A) using the procedures described in example 1 Steps B and C.

Step C: (2S,5S)-4-oxo-5-{[N-(phenylacetyl)-L-isoleucyl]amino}-N-(1H-1,2,3-triazol-4-ylmethyl)-1,2,4,5,6,7-hexahydroazepino[3,2,1-hi]indole-2-carboxamide

The title compound was prepared from (2S,5S)-4-oxo-5-{[N-(phenylacetyl)-L-isoleucyl]amino}-1,2,4,5,6,7-hexahydroazepino[3,2,1-hi]indole-2-carboxylic acid (36 mg, 0.075 mmol, from example 4 Step B) and 1-(1H-1,2,3-triazol-4-yl)methylamine (15 mg, 0.15 mmol, from example 2 Step A) using the procedure described in example 2 Step B). ¹H NMR (500 MHz, DMSO) [symbol] , 8.47 (t, 1H), 8.28 (d, 1H), 8.11 (d, 1H), 7.25 (m, 4H), 7.19 (m, 1H), 7.07 (d, 1H), 7.03 (d, 1H), 6.95 (dd, 1H) 5.01 (d, 1H), 4.20-4.36 (m, 4H), 3.48 (AB, 2H), 3.43 (m 1H), 3.05 (m, 2H), 2.89 (d, 2H), 2.03 (m, 2H), 1.75 (m, 1H), 1.42 (m, 1H), 1.08 (m, 1H), 0.84 (d, 3H), 0.78 (t, 3H). LC-MS (ESI) calc (M+H) 558.2, found. 558.3

### EXAMPLES 5 TO 33

Examples 5 to 33 shown in table 2 were prepared using procedures analogous to those in examples 1 to 4

TABLE 2

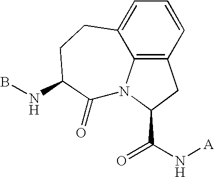

| EXAMPLE # | A | B | LC-MS (ESI) M + H |
|---|---|---|---|
| 5 | 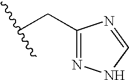 | 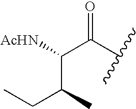 | 482.3 |
| 6 | 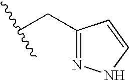 | 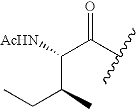 | 481.3 |
| 7 | 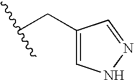 | 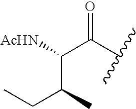 | 481.3 |
| 8 | 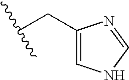 | 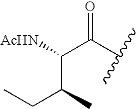 | 481.3 |
| 9 | 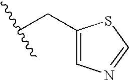 | 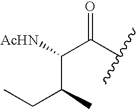 | 498.2 |
| 10 | 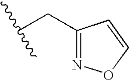 | 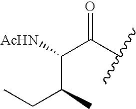 | 482.3 |
| 11 | 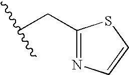 | 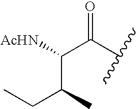 | 498.2 |
| 12 | 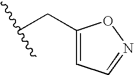 | 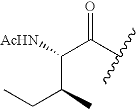 | 482.3 |
| 13 | 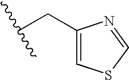 | 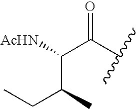 | 498.2 |
| 14 | 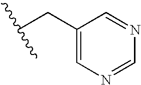 | 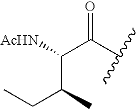 | 493.3 |
| 15 | 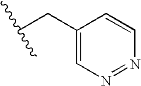 | 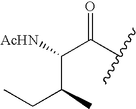 | 493.3 |
| 16 | 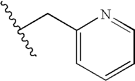 | 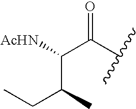 | 492.3 |
| 17 | 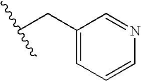 | 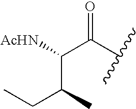 | 492.3 |
| 18 | 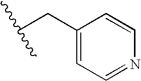 | 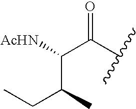 | 492.3 |
| 19 | 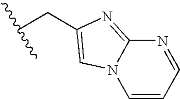 | 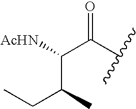 | 532.3 |
| 20 | 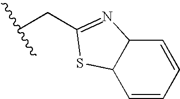 | 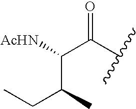 | 548.3 |
| 21 | 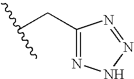 | 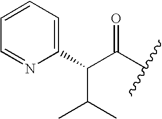 | 489.4 |
| 22 | 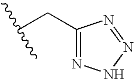 | 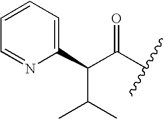 | 489.4 |
| 23 | 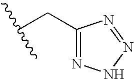 | 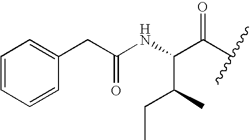 | 559.3 |
| 24 | 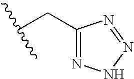 | 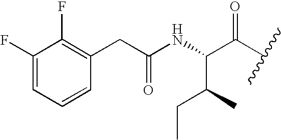 | 595.3 |
| 25 | 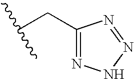 | 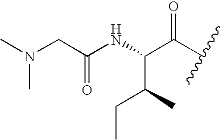 | 526.4 |
| 26 | 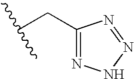 | 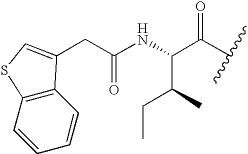 | 615.3 |
| 27 | 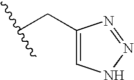 | 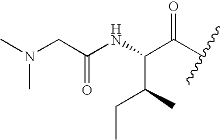 | 525.3 |
| 28 | 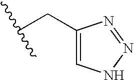 | 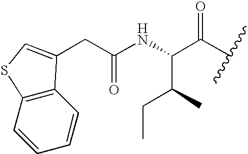 | 614.3 |
| 29 | 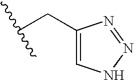 | 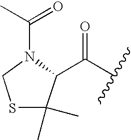 | 512.1 |
| 30 | 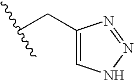 | 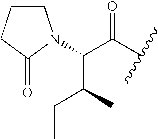 | 508.3 |
| 31 | 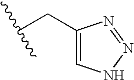 | 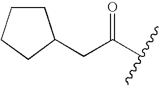 | 437.2 |
| 32 | 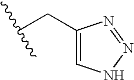 | 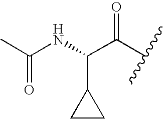 | 466.2 |
| 33 | 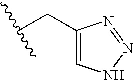 | 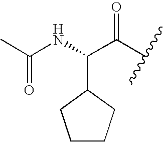 | 494.3 |

Assays for Determining Biological Activity

The Granzyme B inhibitory activity of the compounds of the present invention can be evaluated using the following procedures:

Granzyme B Production.

Plasmids pMelBac and the TA Cloning Kit (pCR2.1) were purchased from Invitrogen (Carlsbad, Calif.). The Bac-to-Bac Baculovirus Expression System and all cell culture supplies were from Life Technologies (Gaithersburg, Md.). The human granzyme B protease has been identified and is known in the art. See, for example, Poe, et al., J. Biol. Chem. 266, 98-103. A vector was generated for secreted protein expression in the Bac-to-Bac system. The honeybee melittin secretion signal was amplified from pMelBac by PCR with primers 5′-GTGT AGA TCT ATG AAA TTC TTA GTC AAC G-3′ and 5′-TTC AGC AGA GTC GAC TCC AAG-3′. The amplified product contained a BglII site upstream of the melittin secretion signal and spanned the multiple cloning site of the vector. The fragment was digested with BglII and EcoRI and subcloned into BamHI and EcoRI digested pFastBac. The resulting vector was designated pSecBac. Activatable tagged human granzyme B was cloned into pSecBac in two sequential steps. Firstly, mature granzyme B was amplified by PCR with primers 5′-GGATCC ATC GAA GGT CGT ATC ATC GGAGGACATGAGGCC-3′ and 5′-AAG CTT TTA GTA GCG CTT CAT GGT CTT CTT TAT CC-3′ and cloned into pCR2.1. In the sense primer, the bold type sequence coding for the Factor Xa recognition site, Ile-Glu-Gly-Arg, was inserted upstream of isoleucine 21 (referred to as ILE 16 in this paper using the numbering scheme for this family of serine proteases is traditionally based on the zymogen, chymotrypsinogen), the first amino acid of mature granzyme B. Secondly, hexa-histidine tagged, activatable granzyme B was amplified from the granzyme B/pCR2.1 clone by PCR using primers 5′-GGA AGA TCT CAT CAT CAT CAT CAT CAT GGA TCC ATC GAA GGT CGT ATC-3′ and 5′-CCT GAA TTC TTA GTA GCG TIT CAT GGT CTT CTT TAT CC-3′. The amplified product contained a Bali site upstream of the hex-histidine tag and contained the Factor Xa recognition sequence. This fragment was digested with Bali and ECOR and subcloned into BamHI and EcoRI digested pSecBac. The completed vector generated a recombinant baculovirus in Gibco BRL's Bac-to-Bac system, which would produce a secreted, 6-histidine tagged granzyme B. For protein expression, sf9 cells were grown to a density of 1.5×10⁶ cells ml⁻¹ in Grace's medium (Cat. 11605-094) supplemented with 10% fetal calf serum and penicillin-streptomycin-glutamine (Cat. 10378-024). Prior to the addition of virus, cells were centrifuged at 1000×g for 15 minutes and resuspended to the same density in fresh growth medium containing recombinant viral stock and SP Sepharose beads (4 ml resin per liter of culture). After 72 hours of induction at 27° C., the resin was collected in a 50 micron Nylon mesh (PGC Scientifics), extensively washed with 50 mM Mes.NaOH, 0.3 M Na Cl, pH 6.6 and poured onto a column. The protein was eluted using a linear gradient (0.3 to 1.0 M) of NaCl. Fractions containing granzyme B activity were identified, combined and concentrated and then diluted in 20 mM Tris.HCl, pH 8.0. The sample was readjusted to a final pH 8.0, CaCl₂ (3 mM final concentration) and factor Xa (Pharmacia, 10 units per mg of protein) were added, and incubated for 18 hours at room temperature to generate active granzyme B. Under these conditions, complete cleavage is achieved. The recombinant granzyme B was purified from the cleavage mixture using a 1 ml HiTrap SP column (Pharmacia). The yield can be as much as 4-5 mg of purified granzyme B per liter of culture. Mass spectral analysis identifies one major peak at 27,466 Da (other components of 27,320 Da and 26,630 Da were also consistently observed). Since the combined mass of the 227-amino acids defined by sequence analysis only accounts for 25,511.58 Da, we concluded that preparations of recombinant granzyme B purified with this method are more homogenous and less glycosylated than preparations of native granzyme B purified from NK cell granules. Nevertheless, the resulting enzyme is indistinguishable from native enzyme with regard to kinetic parameters for inhibition by Ac-IEPD-CHO and other inhibitors.

Fluorogenic Assay for Granzyme B

The activity of granzyme B was measured using a continuous fluorometric assay using the substrate Ac-IEPD-AMC. Briefly, appropriate dilutions of enzyme were added to reaction mixtures containing substrate (10 μM), and various concentrations of the inhibitor of interest in a final reaction volume of 100 μl. Liberation of AMC was monitored continuously at room temperature using a Tecan Fluostar 96-well plate reader (black plates from Dynatech) using an excitation wavelength of 380 nm and an emission wavelength of 460 n m. Unless otherwise indicated, all experiments were carried out at room temperature under standard reaction conditions defined as 0.1 M HEPES, 10% sucrose, 0.1% CHAPS and 10 mM DTT, pH=7.5, 25° C.

Data Analysis

All kinetic constants were computed by direct fits of the data to the appropriate equation using a nonlinear least squares analysis computer program (NLIN). Reaction rates were fit by non-linear regression to the Michaelis-Menton equation for competitive inhibition to obtain a value for the dissociation constant Kᵢ. In cases where inhibition was time-dependent, Kᵢ was instead calculated from the steady-state velocities, or from the rate constants for association (kₒₙ) and dissociation (k_{off}) of enzyme-inhibitor complex according to the equations developed by Morrison for analysis of slow and tight-binding inhibitors (21). The errors in reproducing the rate and dissociation constants were never more than 10%.

Biological Data

The table below shows activity of selected examples. All compounds were inactive against caspase enzymes.

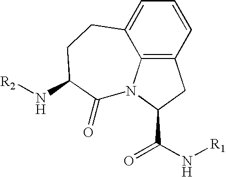

| EXAMPLE # | R₁ | R₂ | Gzm B Kᵢ (nM) |
|---|---|---|---|
| 1 | 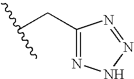 | 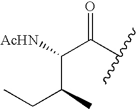 | 74 |
| 2 | 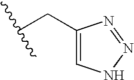 | 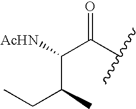 | 38 |
| 3 | 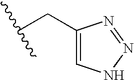 | 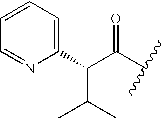 | 75 |
| 4 | 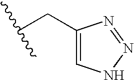 | 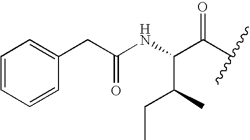 | 13 |
| 19 | 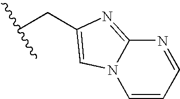 | 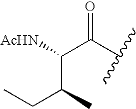 | 788 |
| 21 | 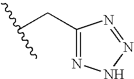 | 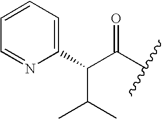 | 410 |
| 23 | 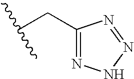 | 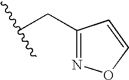 | 22 |
| 24 | 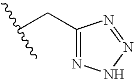 | 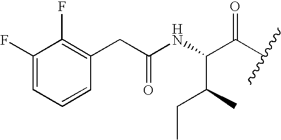 | 36 |
| 25 | 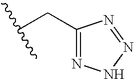 | 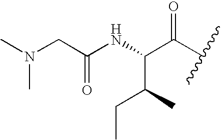 | 203 |
| 26 | 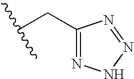 | 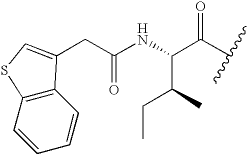 | 17 |
| 27 | 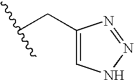 | 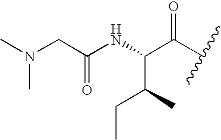 | 45 |
| 28 | 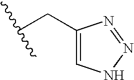 | 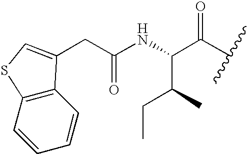 | 7 |
| 29 | 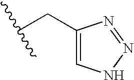 | 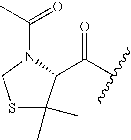 | 3880 |
| 30 | 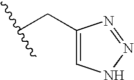 | 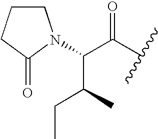 | 112 |
| 31 | 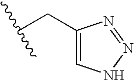 | 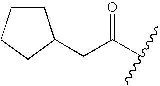 | 1841 |
| 32 | 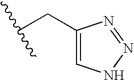 | 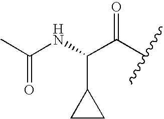 | 1021 |
| 33 | 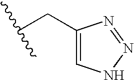 | 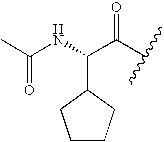 | 71 |

Measurement of Caspase Activity by Cleavage of a Fluorogenic Substrate

Assays for measuring the activity of caspases are well known in the art. For example, the following procedure can be followed to determine the activity of caspase-3:

A fluorogenic derivative of the tetrapeptide recognized by caspase-3 and corresponding to the P₁ to P₄ amino acids of the PARP cleavage site, Ac-DEVD-AMC (AMC amino-4-methylcoumarin) was prepared as follows: i) synthesis of N-Ac-Asp(OBn)-Glu(OBn)-Val-CO₂H, ii) coupling with Asp(OBn)-7-amino-4-methylcoumarin, iii) removal of benzyl groups.

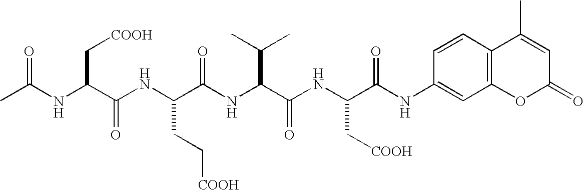

Standard reaction mixtures (300 μL final volume), contained Ac-DEVD-AMC and purified or crude caspase-3 enzyme in 50 mM Hepes/KOH (pH 7.0), 10% (v/v) glycerol, 0.1% (w/v) CHAPS, 2 mM EDTA, 5 mM dithiothreitol, and were incubated at 25° C. Reactions were monitored continuously in a spectrofluorometer at an excitation wavelength of 380 nm and an emission wavelength of 460 nm.

Assays for determining the activity of the other caspases are easily discernible by those skilled in the art.

Data analysis may be performed as described above.

### REFERENCES

The following references are hereby incorporated by reference in their entirety:
Andrade, F., Roy, S., Nicholson, D., Thornberry, N., Rosen, A., and Casciola-Rosen, L. 1998. Granzyme B Directly and Efficiently Cleaves Several Downstream Caspase Substrates: Implications for CTL-Induced Apoptosis Immunity 8:451-460.
Bach, J. F. and S. Koutouzov. (1997). New clues to systemic lupus. Lancet 350:11-11.
Beidler, D. R., Tewari, M., Friesen, P. D., Poirier, G., and Dixit, V. M. (1995). The baculovirus p35 protein inhibits Fas- and tumor necrosis factor-induced apoptosis. J. Biol. Chem. 270, 16526-16528.
Bockenstedt, L. K., R. J. Gee, and M. J. Mamula. (1995). Self-peptides in the initiation of lupus autoimmunity. J. Immunol. 154:3516-3524.
Bump, N. J., Hackett, M., Hugunin, M., Seshagiri, S., Brady, K., Chen, P., Ferenz, C., Franklin, S., Ghayur, T., Li, P., Licari, P., Mankovich, J., Shi, L. F., Greenberg, A. H., Miller, L. K., and Wong, W. W. (1995). Inhibition of ICE family proteases by baculovirus antiapoptotic protein p35. Science 269, 1885-1888.
Burlingame, R. W., R. L. Rubin, R. S. Balderas, and A. N. Theofilopoulos. (1993). Genesis and evolution of antichromatin autoantibodies in murine lupus implicates T-dependent immunization with self-antigen. J. Clin. Invest. 91:1687-1696.
Casciola-Rosen, L. A., Anhalt, G. J., and Rosen, A. (1994a). Autoantigens targeted in systemic lupus erythematosus are clustered in two populations of surface structures on apoptotic keratinocytes. J. Exp. Med. 179, 1317-1330.
Casciola-Rosen, L. A., Miller, D. K., Anhalt, G. J., and Rosen, A. (1994b). Specific cleavage of the 70-kDa protein component of the U1 small nuclear ribonucleoprotein is a characteristic biochemical feature of apoptotic cell death. J. Biol. Chem. 269, 30757-30760.
Casciola-Rosen, L. A., Anhalt, G. J., and Rosen, A. (1995). DNA-dependent protein kinase is one of a subset of autoantigens specifically cleaved early during apoptosis. J. Exp. Med. 182, 1625-1634.
Casciola-Rosen, L. A., Nicholson, D. W., Chong, T., Rowan, K. R., Thornberry, N. A., Miller, D. K., and Rosen, A. (1996). Apoptin/CPP32 cleaves proteins that are essential for cellular repair: A fundamental principle of apoptotic death. J. Exp. Med. 183, 1957-1964.
Casciola-Rosen, L. and A. Rosen. (1997). Ultraviolet light-induced keratinocyte apoptosis: A potential mechanism for the induction of skin lesions and autoantibody production in LE. Lupus 6:175-180.
Casiano, C. A., Martin, S. J., Green, D. R., and Tan, E. M. (1996). Selective cleavage of nuclear autoantigens during CD95 (Fas/APO-1)-mediated T cell apoptosis. J. Exp. Med. 184, 765-770.
Chinnaiyan, A. M. and Dixit, V. M. (1996a). The cell-death machine. Curr. Biol. 6, 555-562.
Chinnaiyan, A. M., Hanna, W. L., Orth, K., Duan, H. J., Poirier, G. G., Froelich, C. J., and Dixit, V. M. (1996b). Cytotoxic T-cell-derived granzyme B activates the apoptotic protease ICE-LAP3. Curr. Biol. 6, 897-899.
Darmon, A. J., Ley, T. J., Nicholson, D. W., and Bleackley, R. C. (1996). Cleavage of CPP32 by granzyme B represents a critical role for granzyme B in the induction of target cell DNA fragmentation. J. Biol. Chem. 271, 21709-21712.
Darmon, A. J., Nicholson, D. W., and Bleackley, R. C. (1995). Activation of the apoptotic protease CPP32 by cytotoxic T-cell-derived granzyme B. Nature 377, 446-448.
Deveraux, Q. L., Takahashi, R., Salvesen, G. S. and Reed, J. C. (1997). X-linked JAP is a direct inhibitor of cell-death proteases. Nature 38, 300-304.
Diamond, B., J. B. Katz, E. Paul, C. Aranow, D. Lustgarten, and M. D. Scharff. (1992). The role of somatic mutation in the pathogenic anti-DNA response. Ann. Rev. Immunol. 10:731-757.
Duan, H., Orth, K., Chinnaiyan, A. M., Poirier, G. G., Froelich, C. J., He, W.-W., and Dixit, V. M. (1996). ICE-LAP6, a novel member of the ICE/Ced-3 gene family, is activated by the cytotoxic T cell protease granzyme B. J. Biol. Chem. 271, 16720-16724.
Fernandes-Alnem-ri, T., Armstrong, R. C., Krebs, J., Srinivasula, S. M., Wang, L., Bullrich, F., Fritz, L. C., Trapani, J. A., Tomaselli, K. J., Litwack, G., and Alnemri, E. S. (1996). In vitro activation of CPP32 and Mch.3 by Mch4, a novel human apoptotic cysteine protease containing two FADD-like domains. Proc. Natl. Acad. Sci. USA 93, 7464-7469.
Froelich, C. J., Hanna, W. L., Poirier, G. G., Duriez, P. J., D'Amours, D., Salvesen, G. S., Alnemri, E. S., Earnshaw, W. C., and Shah, G. M. (1996a). Granzyme B perforin-mediated apoptosis of jurkat cells results in cleavage of poly(ADP-ribose) polymerase to the 89-kDa apoptotic fragment and less abundant 64-kDa fragment. Biochem. Biophys. Res. Commun. 227, 658-665.
Froelich, C. J., Orth, K., Turbov, J., Seth, P., Gottlieb, R., Babior, B., Shah, G. M., Bleackley, R. C., Dixit, V. M., and Hanna, W. (1996b). New paradigm for lymphocyte granule-mediated cytotoxicity—Target cells bind and internalize granzyme B, but an endosomolytic agent is necessary for cytosolic delivery and subsequent apoptosis. J. Biol. Chem. 271, 29073-29079.
Ghayur, T., Hugunin, M., Talanian, R. V., Ratnofsky, S., Quinlan, C., Emoto, Y., Pandy, P., Datta, R., Huang, Y., Kharbanda, S., Allen, H., Kamen, R., Wong, W., and Kufe, D. (1996). Proteolytic activation of protein kinase C d by an ICE/CED-3-like protease induces features of apoptosis. J. Exp. Med. 184, 2399-2404.
Greidinger, E. L., Miller, D. K., Yamin, T.-T., Casciola-Rosen, L., and Rosen, A. (1996). Sequential activation of three distinct ICE-like activities in Fas-ligated Jurkat cells. FEBS Lett. 390, 299-303.
Gu, Y., Sarnecki, C., Fleming, M. A., Lippke, J. A., Bleakley, R. C., and Su, M. S. S. (1996). Processing and Activation of CMH-1 by Granzyme B. J. Biol. Chem. 271, 10816-10820.
Heusel, J W., Wesselschmidt, R. L., Shresta, S., Russell, J. H., and Ley, T. J. (1994). Cytotoxic lymphocytes require granzyme B for the rapid induction of DNA fragmentation and apoptosis in allogeneic target cells. Cell 76, 977-987.
Irmler, M., Thome, M., Hahne, M., Schneider, P., Hofmann, B., Steiner, V., Bodmer, J. L., Schr6ter, M., Burns, K., Mattmann, C., Rimoldi, D., French, L. E., and Tschopp, J. (1997). Inhibition of death receptor signals by cellular FLIP. Nature 388, 190-195.
Jacobson, M. D., Weil, M., and Raff, M. C. (1997). Programmed cell death in animal development. Cell 88, 347-354.
Jans, D. A., Jans, P., Briggs, L. J., Sutton, V., and Trapani, J. A. (1996). Nuclear transport of granzyme B (fragmentin 2). Dependence on perforin in vivo and cytosohe factors in vitro. J. Biol. Chem. 271, 30781-30789.
Krajewska, M., Wang, H. G., Krajewski, S., Zapata, J. M., Shabaik, A., Gascoyne, R., and Reed, J. C. (1997). Immunohistochemical analysis of in vivo patterns of expression of CPP32 (Caspase-3), a cell death protease. Cancer Res. 57, 1605-1613.
Krajewski, S., Gascoyne, R. D., Zapata, J. M., Krajewska, M., Kitada, S., Chanabhai, M., Horsman, D., Berean, K., Piro, L. D., Fugier-Vivier, I., Liu, Y. J., Wang, H. G., and Reed, J. C. (1997). Immunolocalization of the ICE/Ced-3-family protease, CPP32 (Caspase-3), in non-Hodgkin's lymphomas, chronic lymphocytic leukemias, and reactive lymph nodes. Blood 89, 3817-3825.
Lanzavecchia, A. (1995). How can cryptic epitopes trigger autoimmunity? J. Exp. Med. 181, 1945-1948.
Liu, X., Zou, H., Slaughter, C., and Wang, X. (1997). DFF, a heterodimeric protein that functions downstream of caspase-3 to trigger DNA fragmentation during apoptosis. Cell 89, 175-184.
Mamula, M. J. (1993). The inability to process a self-peptide allows autoreactive T cells to escape tolerance. J. Exp. Med. 177:567-571.
Martin, S. J., Amarante-Mendes, G. P., Shi, L. F., Chuang, T. H., Casiano, C. A., O'Brien, G. A., Fitzgerald, P., Tan, E. M., Bokoch, G. M., Greenberg, A. H., and Green, D. R. (1996). The cytotoxic cell protease granzyme B initiates apoptosis in a cell-free system by proteolytic processing and activation of the ICE/CED-3 family protease, CPP32, via a novel two-step mechanism. EMBO J. 15, 2407-2416.
Martin, S. J. and Green, D. R. (1995). Protease activation during apoptosis: Death by a thousand cuts? Cell 82, 349-352.
McGahon, A. J., Nishioka, W. K., Martin, S. J., Mahboubi, A., Cotter, T. G. and Green, D. R. (1995). Regulation of the Fas apoptotic cell death pathway by Abl. J. Biol. Chem. 270, 22625-22631.
Muzio, M., Chinnaiyan, A. M., Kischkel, F. C., O'Rourke, K., Shevchenko, A., Ni, J., Scaffidi, C., Bretz, J. D., Zhang, M., Gentz, R., Mann, M., Krammer, P. H., Peter, M. E., and Dixit, V. M. (1996). FLICE, a novel FADD-homologous ICE/CED-3-like protease, is recruited to the CD95 (Fas/APO-1) death-inducing signaling complex. Cell 85, 817-827.
Nicholson, D. W., Ali, A., Thornberry, N. A., Vaillancourt, J. P., Ding, C. K., Gallant, M., Gareau, Y., Griffin, P. R., Labelle, M., Lazebnik, Y. A., Munday, N. A., Raju, S. M., Smulson, M. E., Yamin, T., Yu, V. L., and Miller˜ D. K. (1995). Identification and inhibition of the ICE/CED-3 protease necessary for mammalian apoptosis. Nature 376, 37-43.
Nicholson, D. W. and Thornberry, N. A. (1997). Caspases: Killer proteases. TIBS 22, 299-306.
Odake, S., Kam, C. M., Narasimhan, L., Poe, M., Blake, J. T., Krahenbuhl, O, Tschopp, J., and Powers, J. C. (1991). Human and murine cytotoxic T lymphocyte serine proteases: Subsite mapping with peptide thioester substrates and inhibition of enzyme activity and cytolysis by isocoumarins. Biochemistry 30, 2217-2227.
Pinkoski, M. J., Winkler, U., Hudig, D., and Bleackley, R. C. (1996). Binding of granzyme B in the nucleus of target cells. Recognition of an 80 kDa protein. J. Biol. Chem. 271, 10225-10229.
Podack, E. and Konigsberg, P. J. (1984). Cytolytic T cell granules. Isolation, structural, biochemical and functional characterization. J. Exp. Med. 160, 695-710.
Poe, M., Blake, J. T., Boulton, D. A., Gammon, M., Sigal, N. H., Wu, J. K., and Zweerink, H. J. (1991). Human cytotoxic lymphocyte granzyme B: Its purification from granules and the characterization of substrate and inhibitor specificity. J. Biol. Chem. 266, 98-103.
Quan, L. T., Tewari, M., O'Rourke, K., Dixit, V., Snipas, S. J., Poirier, G. G., Ray, C., Pickup, D. J., and Salveson, G. S. (1996). Proteolytic activation of the cell death protease Yama/CPP32 by granzyme B. Proc. Natl. Acad. Sci. USA. 93, 1972-1976.
Radic, M X and M. Weigert. (1994). Genetic and structural evidence for antigen selection of anti-DNA antibodies. Ann. Rev. Immunol. 12:487-520.
Ramage, P., Cheneval, D., Chvei, M., Graff, P., Hemmig, R., Heng, R., Kocher, H. P., Mackenzie, A., Memmert, K., Revesz, L., and Wishart, W. (1995). Expression, refolding, and autocatalytic proteolytic processing of the interleukin-1b-converting enzyme precursor. J. Biol. Chem. 270, 9378-9383.
Salemi, S., A. P. Caporossi, L. Boffa, M. G. Longobardi, and V. Barnaba. (1995). HIVgp12O activates autoreactive CD4-specific T cell responses by unveiling of hidden CD4 peptides during processing. J. Exp. Med. 181:2253-2257.
Sarin, A., Williams, M. S., Alexander-Miller, M. A., Berzofsky, J. A., Zacharchuk, C. M., and Henkart, P. A. (1997). Target cell lysis by CTL granule exocytosis is independent of ICE/Ced-3 family proteases. Immunity 6:209-215.
Sercarz, E. E., P. V. Lehmann, A. Ametani, G. Benichou, A. Miller, and K. Moudgil. (1993). Dominance and crypticity of T cell antigenic determinants. Ann. Rev. Immunol. 11:729-766.
Sercarz, E. E. and S. K. Datta. (1994). Mechanisms of autoimmunization: perspective from the mid-90s. Curr. Opin. Immunol. 6:875-881.
Shi, L. F., Mai, S., Israel, S., Browne, K., Trapani, J. A., and Greenberg, A. H. (1997). Granzyme B (GraB) autonomously crosses the cell membrane and perforin initiates apoptosis and GraB nuclear localization. J. Exp. Med. 185, 855-866.
Shresta, S., MacIvor, D. M., Heusel, J. W., Russell, J. H., and Ley, T. J. (1995). Natural killer and lympholdne-activated killer cells require granzyme B (ˆ) for the rapid induction of apoptosis in susceptible target cells. Proc. Natl. Acad. Sci. USA 92, 5679-5683.
Simitsek, P. D., D. G. Campbell, A. Lanzavecchia, N. Fairweather, and C. Watts. (1995). Modulation of antigen processing by bound antibodies can boost or suppress class II major histocompatibility complex presentation of different T cell determinants. J. Exp. Med. 181:1957-1963.
Song, Q. Z., Burrows, S. R., Smith, G., Lees-Miller, S. P., Kumar, S., Chan, D. W., Trapani, J. A., Alnemri, E., Litwack, G., Lu, H., Moss, D. J., Jackson, S., and Lavin, MY (1996a). Interleukin-1b-converting enzyme-like protease cleaves DNA-dependent protein kinase in cytotoxic T cell killing. J. Exp. Med. 184, 619-626.
Song, Q. Z., Lees-Miller, S. P., Kumar, S., Zhang, N., Chan, D. W., Smith, G. C. M., Jackson, S. P., Alnemri, E. S., Litwack, G., Khanna, K. K., and Lavin, M. F. (1996b). DNA-dependent protein kinase catalytic subunit: A target for an ICE-like protease in apoptosis. EMBO J. 15, 3238-3246.
Srinivasula, S. M., Fernandes-Alnem-ri, T., Zangrilli, J., Robertson, N., Armstrong, R. C., Wang, L. J., Trapani, J. A., Tomaselli, K. J., Litwack, G., and Alnemri, E. S. (1996). The Ced-3/interleukin 1b converting enzyme-like homolog Mch.6 and the lamin-cleaving enzyme Mch2a are substrates for the apoptotic mediator CPP32. J. Biol. Chem. 271, 27099-1027106.
Srinivasula, S. M., Ahmad, M., Ottilie, S., Bullrich, F., Banks, S., Wang, Y., Fernandes-Alnem-ri, T., Croce, C. M., Litwack, G., Tomaselli, K. J., Armstrong, R. C and Alnemri, E. S. (1997). FLAME-1, a novel FADD-like anti-apoptotic molecule that regulates Fas/TNFR1-induced 15apoptosis. J. Biol. Chem. 272, 18542-18545.
Talanian, R. V., Yang, X., Turbov, J., Seth, P., Ghayur, T., Casiano, C. A., Orth, K., and Froelich, C. J. (1997). Granule-mediated killing: Pathways for granzyme B-initiated apoptosis. J. Exp. Med. 186, 1323-1331.
Thome, M., Schneider, P., Hofmann, K., Fickenscher, H., Meinl, E., Neipel, F., Mattmann, C., Burns, K., Bodmer, J. L., Schr6ter, M., Scaffidi, C., Krammer, P. H., Peter, M. E., and Tschopp, J. (1997). Viral FLICE-inhibitory proteins (Fs) prevent apoptosis induced by death receptors. Nature 386, 517-521.
Thompson, C. B. (1995). Apoptosis in the pathogenesis and treatment of disease. Science 267, 1456-1462.
Thornberry, N. A. and Molineaux, S. M. (1995). Interleukin-1 B converting enzyme: a novel cysteine protease required for IL-1 13 production and implicated in programmed cell death. Protein Science 4, 3-12.
Thornberry, N. A., Ranon, T. A., Pieterson, E. P., Rasper, D. M., Timkey, T., Garcia-Calvo, M., Houtzager, V. M., Nordstrom, P. A., Roy, S., Vaillancourt, J. P., Chapman, K. T., and Nicholson, D. W. (1997). A combinatorial approach defines specificities of members of the caspase family and granzyme B—Functional, relationships established for key mediators of apoptosis. J. Biol. Chem 272, 17907-17911.
Topalian, S. L., Solomon, D. and Rosenberg, S. A. (1989). Tumor-specific cytolysis by lymphocytes infiltrating human melanomas. J. Immunol. 142, 3714-3725.
Trapani, J. A., Browne, K. A., Smyth, M. J., and Jans, D. A. (1996). Localization of granzyme B in the nucleus—A putative role in the mechanism of cytotoxic lymphocyte-mediated apoptosis. J. Biol. Chem. 271, 4127-4133.
Tschopp, J. (1994). Granzyme B. Methods Enzymol. 244, 80-87.
Wang, S. Y., Miura, M., Jung, Y. K., Zhu, H., Gagliardini, V., Shi, L. F., Greenberg, A. H., and Yuan, J. Y. (1996). Identification and characterization of Ich-3, a member of the interleukin-1b converting enzyme (ICE)/Ced-3 family and an upstream regulator of ICE. J. Biol. Chem. 271, 20580-20587.
Watts, C. and A. Lanzavecchia. (1993). Suppressive effect of an antibody on processing of T cell epitopes. J. Exp. Med. 178:1459-1463.
White, E. (1996). Life, death, and the pursuit of apoptosis. Genes Dev. 10, 1-15.
Xue, D. and Horvitz, H. R. (1995). Inhibition of the Caenorhabditis elegans cell-death protease CED-3 by a CED-3 cleavage site in baculovirus p35 protein. Nature 377, 248-251.
Yamin, T. T., Ayala, J. M., and Miller, D. K. (1996). Activation of the native 45-kDa precursor form of interleukin-1B-converting enzyme. J. Biol. Chem. 271, 13273-13282.
Young, J. D.-E., Hengartner, H., Podack, E., and Cohn, Z. A. (1986). Purification and characterization of a cytolytic pore-forming protein from granules of cloned lymphocytes with natural killer activity. Cell 44, 849-859.

What is claimed is:

1. A compound represented by Formula I: 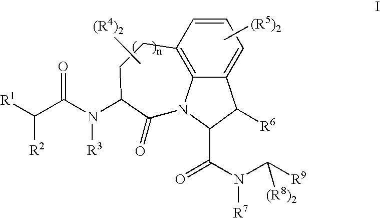
or a pharmaceutically acceptable salt or hydrate thereof, wherein:
n is 0, 1, or 2;
R¹ and R² are each independently selected from the group consisting of: hydrogen, C₁₋₆alkyl, C₁₋₆alkoxy, C₃₋₆cycloalkyl, aryl, HET and —N(R¹⁰)₂, wherein:
(a) said C₁₋₆alkyl, C₁₋₆alkoxy and C₃₋₆cycloalkyl are optionally substituted with 1-3 substituents independently selected from the group consisting of halo and hydroxy; and
(b) said aryl and BET are optionally substituted with 1-3 substituents independently selected from the group consisting of: halo, hydroxy and C₁₋₄alkyl, optionally substituted with 1-3 halo groups;
or R1 and R2 may be joined together with the carbon atom to which they are attached to form a five or six membered monocyclic ring, optionally containing 1-3 heteroatoms selected from the group consisting of: S, O and N(R¹⁰), wherein said ring is optionally substituted with 1-3 R¹⁰ groups,
with the proviso that R¹ and R² are both not hydrogen;
each of R³ and R⁷ is independently selected from the group consisting of: hydrogen and C₁₋₄allyl, optionally substituted with 1-3 halo groups;
each of R⁴, R⁵, R⁶ and R⁸ is independently selected from the group consisting of: hydrogen, halo, hydroxy and C₁₋₄alkyl, optionally substituted with 1-3 halo groups;
R⁹ is HET, optionally substituted with 1-3 substituents independently selected from the group consisting of: halo, hydroxy and C₁₋₄allyl, optionally substituted with 1-3 halo groups;
R¹⁰ is selected from the group consisting of: hydrogen, C₁₋₄alkyl and —C(O)C₁₋₄alkyl, said —C(O)C₁₋₄alkyl optionally substituted with N(R¹¹)₂, HET and aryl, said aryl optionally substituted with 1-3 halo groups;
R¹¹ is selected from hydrogen and C₁₋₄alkyl, optionally substituted with 1-3 halo groups;
HET is a 5- to 10-membered aromatic, partially aromatic or non-aromatic mono- or bicyclic ring, containing 1-4 heteroatoms selected from O, S and N(R¹²), and optionally substituted with 1-2 oxo groups; and
R¹² is selected from the group consisting of: hydrogen and C₁₋₄alkyl, optionally substituted with 1-3 halo groups.

2. The compound according to claim 1 wherein n is 0.

3. The compound according to claim 1 wherein n is 1.

4. The compound according to claim 1 wherein n is 2.

5. The compound according to claim 1 wherein each of R³, R⁴, R⁵, R⁶, R⁷ and R⁸ is hydrogen.

6. The compound according to claim 1 wherein HET is selected from the group consisting of: benzimidazolyl, benzofuranyl, benzopyrazolyl, benzotriazolyl, benzothiophenyl, benzoxazolyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, tetrazolyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, tetrahydrofuranyl, and tetrahydrothienyl, each optionally substituted with 1-2 substituents independently selected from the group consisting of: halo, oxo, hydroxy and C₁₋₄alkyl, optionally substituted with 1-3 halo groups.

7. The compound according to claim 1 wherein R⁹ is selected from the group consisting of: pyridyl, pyrimidyl, pyrazinyl, pyridazinyl, imidazolyl, pyrazolyl, thiazolyl, oxazolyl, oxadiazolyl, isoxazolyl, 1,2,4-triazolyl, 1,2,3-triazolyl, and tetrazolyl, each optionally substituted with 1-3 substituents independently selected from the group consisting of: halo, hydroxy and C₁₋₄alkyl, optionally substituted with 1-3 halo groups.

8. The compound according to claim 1 wherein R¹ and R² are each independently selected from the group consisting of: C₁₋₆alkyl, C₃₋₆cycloalkyl, phenyl, pyridyl, 2-oxopyrrolidine and —N(R¹⁰)₂, wherein:
(a) said C₁₋₆alkyl and C₃₋₆cycloalkyl optionally substituted with 1-3 groups independently selected from the group consisting of halo and hydroxy; and
(b) said phenyl, pyridyl and 2-oxopyrrolidine optionally substituted with 1-3 groups independently selected from the group consisting of: halo, hydroxy and C₁₋₄alkyl, optionally substituted with 1-3 halo groups; and
R¹⁰ is selected from the group consisting of: hydrogen, C₁₋₄alkyl, and —C(O)C₁₋₄alkyl, said —C(O)C₁₋₄alkyl optionally substituted with N(R¹¹)₂, pyrrolidine, piperidine, morhpoline, benzothiophene and phenyl, said phenyl optionally substituted with 1-3 halo groups.

9. The compound according to claim 8 wherein n is 1.

10. The compound according to claim 9 wherein each of R³, R⁴, R⁵, R⁶, R⁷ and R⁸ is hydrogen.

11. The compound according to claim 10 wherein R⁹ is selected from the group consisting of: pyridyl, pyrimidyl, pyrazinyl, pyridazinyl, imidazolyl, pyrazolyl, thiazolyl, oxazolyl, oxadiazolyl, isoxazolyl, 1,2,4-triazolyl, 1,2,3-triazolyl, and tetrazolyl, each optionally substituted with 1-3 substituents independently selected from the group consisting of: halo, hydroxy and C₁₋₄alkyl, optionally substituted with 1-3 halo groups.

12. The compound according to claim 1 of Formula II: 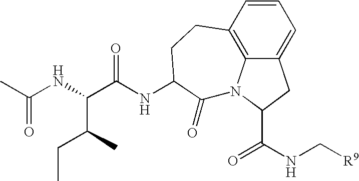
or a pharmaceutically acceptable salt or hydrate thereof, wherein:
R⁹ is selected from the group consisting of: benzimidazolyl, benzofuranyl, benzopyrazolyl, benzotriazolyl, benzothiophenyl, benzoxazolyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, tetrazolyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, tetrahydrofuranyl, and tetrahydrothienyl.

13. The compound according to claim 12 wherein R⁹ is selected from the group consisting of: pyridyl, pyrimidyl, pyrazinyl, pyridazinyl, imidazolyl, pyrazolyl, thiazolyl, oxazolyl, oxadiazolyl, isoxazolyl, 1,2,4-triazolyl, 1,2,3-triazolyl, and tetrazolyl.

14. A compound selected from the group consisting of:

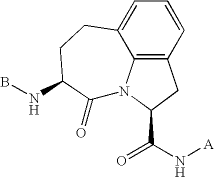

| A | B |
|---|---|
| 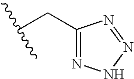 | 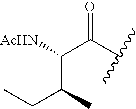 |
| 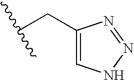 | 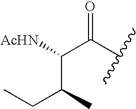 |
| 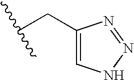 | 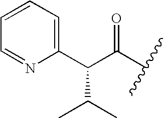 |
| 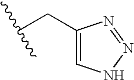 | 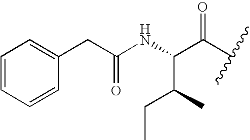 |
| 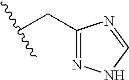 | 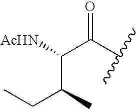 |
| 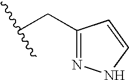 | 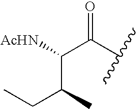 |
| 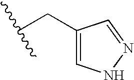 | 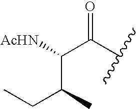 |
| 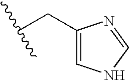 | 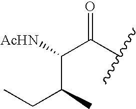 |
| 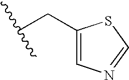 | 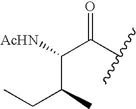 |
| 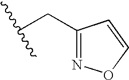 | 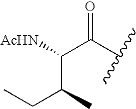 |
| 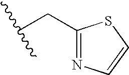 | 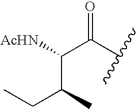 |
| 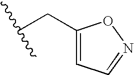 | 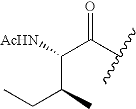 |
| 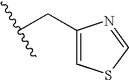 | 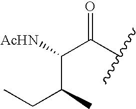 |
| 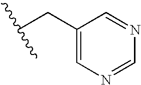 | 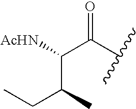 |
| 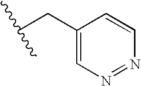 | 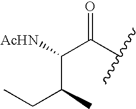 |
| 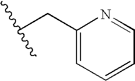 | 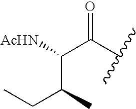 |
| 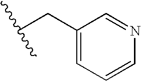 | 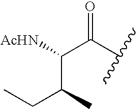 |
| 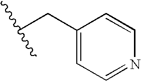 | 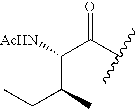 |
| 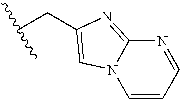 | 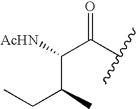 |
| 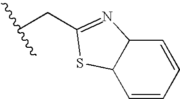 | 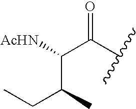 |
| 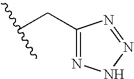 | 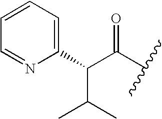 |
| 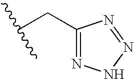 | 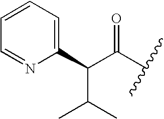 |
| 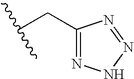 | 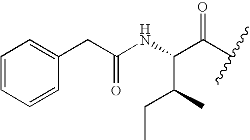 |
| 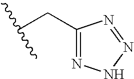 | 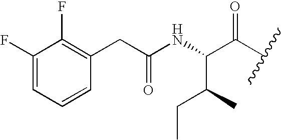 |
| 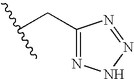 | 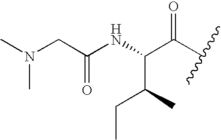 |
| 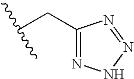 | 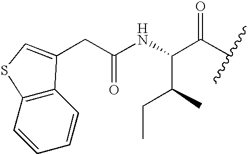 |
| 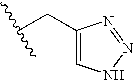 | 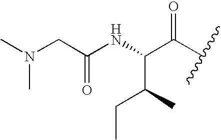 |
| 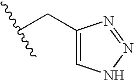 | 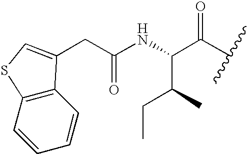 |
| 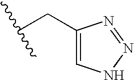 | 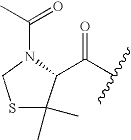 |
| 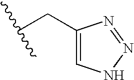 | 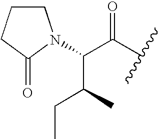 |
| 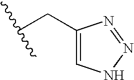 | 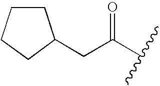 |
| 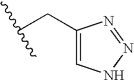 | 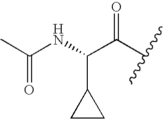 |
| 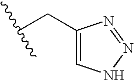 | 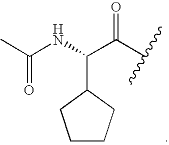 |

15. A pharmaceutical composition comprising a compound in accordance with claim 1 in combination with a pharmaceutically acceptable carrier.

16. A method of treating an immunoregulatory abnormality in a mammalian patient in need of such treatment comprising administering to said patient a compound in accordance with claim 1 in an amount that is effective for treating said immunoregulatory abnormality.

17. The method according to claim 16 wherein the immunoregulatory abnormality is an autoimmune or chronic inflammatory disease selected from the group consisting of: systemic lupus erythematosis, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmopathy, asthma, schleroderma and Sjogren's syndrome.

18. The method according to claim 16 wherein the immunoregulatory abnormality is bone marrow or organ transplant rejection or graft-versus-host disease.

19. The method according to claim 16 wherein the immunoregulatory abnormality is selected from the group consisting of: transplantation of organs or tissue, graft-versus-host diseases brought about by transplantation, autoimmune syndromes including rheumatoid arthritis, systemic lupus erythematosus, Hashimoto's thyroiditis, multiple sclerosis, myasthenia gravis, type I diabetes, uveitis, posterior uveitis, allergic encephalomyelitis, glomerulonephritis, post-infectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C₄ release, Behcet's disease, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, trauma, chronic bacterial infection, malignancy of lymphoid origin and acute and chronic lymphocytic leukemias and lymphomas.

20. A method of suppressing the immune system in a mammalian patient in need of immunosuppression comprising administering to said patient an immunosuppressing effective amount of a compound of claim 1.

21. A pharmaceutical composition comprising a compound which inhibits granzme B and does not substantially inhibit any caspase protease in combination with a pharmaceutically acceptable carrier.

22. The pharmaceutical composition in accordance with claim 21 comprising a compound which possesses a Ki of 500 nM or less for inhibiting granzyme B and possesses a Ki of 10,000 nM or more for inhibiting each of caspase-1 to caspase-13 in combination with a pharmaceutically acceptable carrier.

23. A method of treating an immunoregulatory abnormality in a mammalian patient in need of such treatment comprising administering to said patient a compound which inhibits granzme B and does not substantially inhibit any caspase protease in an amount that is effective for treating said immunoregulatory abnormality.

24. The method of treating an immunoregulatory abnormality in a mammalian patient in need of such treatment in accordance with claim 23 comprising administering to said patient a compound which possesses a Ki of 500 nM or less for inhibiting granzyme B and possesses a Ki of 10,000 nM or more for inhibiting each of caspase-1 to caspase-13 in an amount that is effective for treating said immunoregulatory abnormality.
